(19) United States
(12) Patent Application Publication
(10) Pub. No.: US 2025/0075003 A1
(43) Pub. Date: Mar. 6, 2025

(54) NANOPARTICLES COMPRISING FUSION PROTEIN OF SINGLE-CHAIN VARIABLE FRAGMENT AND FERRITIN, AND USE THEREOF

(71) Applicants: UIF (University Industry Foundation), Yonsei University, Seoul (KR); RESEARCH & BUSINESS FOUNDATION SUNGKYUNKWAN UNIVERSITY, Suwon-si, Gyeonggi-do (KR)

(72) Inventors: Baik Lin SEONG, Seoul (KR); Sangjeon CHUNG, Suwon-si (KR); Min Jin KIM, Yeonsu-gu (KR); Hyo Jin KANG, Suwon-si (KR); Ga Hyeon KIM, Suwon-si (KR); Young Seok KIM, Seoul (KR); Je Ho LEE, Gimpo-si (KR); Myunghyun SOHN, Seoul (KR); Tae Hoon Kim, Yeonsu-gu (KR)

(73) Assignees: UIF (University Industry Foundation), Yonsei University, Seoul (KR); RESEARCH & BUSINESS FOUNDATION SUNGKYUNKWAN UNIVERSITY, Suwon-si, Gyeonggi-do (KR)

(21) Appl. No.: 18/559,548

(22) PCT Filed: May 9, 2022
(86) PCT No.: PCT/KR2022/006597

§ 371 Date: Jun. 10, 2024

(30) Foreign Application Priority Data
May 7, 2021 (KR) 10-2021-0059544

(51) Int. Cl. C07K 16/32; A61K 9/14; A61K 38/00; C07K 14/47; C07K 16/22; C12N 9/00

(52) U.S. Cl. CPC C07K 16/32; A61K 9/14; C07K 14/47; C07K 16/22; C12N 9/93; C12Y 601/01006; A61K 38/00; C07K 2317/24; C07K 2317/622; C07K 2319/30

(57) ABSTRACT

The present invention provides: ferritin; a single-chain variable fragment bound to the N-terminus of the ferritin; and a fusion protein self-assembling nanoparticle structure in which an N-terminus RNA-interaction domain (RID) in a human-derived lysyl-tRNA synthetase is bound to the N-terminus of the single-chain variable fragment by means of a novel fusion partner. Therefore, the present invention provides: a fusion protein having enhanced water solubility; nanoparticles; a vector coding same; a host cell transformed by means of the vector, and a pharmaceutical composition, for treating a disease, using the same.

### CROSS-REFERENCE TO RELATED APPLICATION

This application claims priority to and the benefit of Korean Patent Application No. 10-2021-0059544, filed on May 7, 2021, the disclosure of which is incorporated herein by reference in its entirety.

### TECHNICAL FIELD

The present invention relates to ferritin, a fusion protein including a single-chain variable fragment linked to the N-terminus of the ferritin and an N-terminus domain RID (RNA-interaction domain) of a human-derived lysyl tRNA synthetase linked to the N-terminus of the single-chain variable fragment, self-assembled nanoparticles of the fusion protein, an expression vector including a nucleotide for encoding the fusion protein, and novel uses thereof. The fusion protein and nanoparticles of the present invention have the advantage of greatly enhancing water solubility by using RID, and by varying the origin of single-chain variable fragments, it is possible to prepare a pharmaceutical composition for treating a desired disease.

### BACKGROUND ART

Viruses are largely divided into two types: enveloped viruses and non-enveloped viruses. In the case of non-enveloped viruses, the viral surface consists only of capsid proteins, and therefore, self-assembly into VLPs is possible only with capsid proteins. However, enveloped viruses essentially require a membrane component to form a virus. Therefore, for self-assembly into virus-like particles (VLPs), membrane components are required in addition to surface antigens. That is, in the envelope-virus-like particle (VLP) self-assembly process, not only the folding of the monomeric antigen protein but also the lipid membrane thereof is essentially required. However, the lipid membrane is structurally non-uniform and difficult to characterize, and thus, the formation of VLPs could only be generated in higher eukaryotic cells (animal cells, insect cells, human-derived cells, yeast, etc.). Therefore, there is a need for a method for commonly constructing various antigenic proteins by the recombination and expression of a self-assembling protein as a scaffold replacing a membrane component.

Ferritin is present in most biological species and has the characteristic of self-assembling to form nanoparticles. In previous studies, viral nanoparticles were produced in eukaryotic cells (insects and animal cells) by using human-derived ferritin or Helicobacter pylori ferritin (L. He, N. de Val et al.: Presenting nativelike trimeric HIV-1 antigens with self-assembling nanoparticles. Nat Commun, 7, 12041 (2016) doi: 10.1038/ncomms12041, H. M. Yassine et al.: Hemagglutinin-stem nanoparticles generate heterosubtypic influenza protection. Nature Medicine, 21 (9), 1065-1070 (2015)). However, the eukaryotic cell production system has problems such as high cost, low productivity and difficult mass production process.

In order to solve these problems, it is necessary to develop a method for producing a ferritin nanoparticle structure using E. coli. However, when the foreign protein is expressed in E. coli, there is a problem in that the protein is difficult to fold, and thus, problems have occurred in which the cost and time increase due to the additional requirement of a refolding process.

The inventors of the present invention intend to solve the above problems and use the nanoparticles prepared therefrom as a disease-specific therapeutic agent.

### DISCLOSURE

### Technical Problem

In order to solve the problems of the related art, an object of the present invention is to provide a recombinant expression vector that can produce water-soluble single-chain variable fragments that are difficult to express in E. coli in a water-soluble form and self-assemble to form stable nanoparticles, and a host cell which is transformed by the expression vector.

Another object of the present invention is to provide a method for preparing a water-soluble fusion protein and nanoparticles using the recombinant expression vector and host cell.

Still another object of the present invention is to provide a pharmaceutical composition for preventing, ameliorating or treating disease, including the nanoparticles prepared by the above preparation method.

### Technical Solution

The inventors of the present invention used the RNA-interaction domain (RID) among human-derived lysyl tRNA synthetases at the N-terminus of a single-chain variable fragment as a novel fusion partner, and fabricated a fusion protein in which the single-chain variable fragment is bound to an N-terminus of ferritin, nanoparticles in which the fusion protein is self-assembled and an expression vector thereof, and by using the same, insoluble misfolded single-chain variable fragments, which were previously difficult to produce in a water-soluble form, were produced in a properly folded water-soluble form without refolding.

That is, according to an exemplary embodiment of the present invention, provided are nanoparticles, including a ferritin protein; a single-chain variable fragment which is bound to an N-terminus of the ferritin protein; and an N-terminus domain (hLysRS N-terminal appended RNA interacting domain; hRID) which is isolated from a human-derived lysyl tRNA synthetase that is bound to an N-terminus of the single-chain variable fragment, and also provided is a recombination expression vector including a polynucleotide for encoding the nanoparticles.

The type of the single-chain variable fragment of the present invention is not limited, but most preferably, it is derived from any one antibody selected from the group consisting of Trastuzumab, Bevacizumab and Pertuzumab.

According to the present invention, the hRID may be represented by the amino acid sequence of SEQ ID NO: 1, and the ferritin protein may be represented by the amino acid sequence of SEQ ID NO: 4.

The vector may include a linker between the polynucleotide encoding the single-chain variable fragment (scFv) and the polynucleotide encoding the ferritin. The linker may be represented by an amino acid sequence represented by any one of the nucleotide sequences of SEQ ID NOs: 8 to 10.

In addition, the present invention provides a host cell, which is transformed by the expression vector. In the present invention, for the host cell, any host cell that are readily available to those skilled in the art to which the present invention pertains may be used without limitation, but preferably, the host cell may be selected from the group consisting of Escherichia bacteria, Bacillus bacteria, Pseudomonas bacteria, lactic acid bacteria, yeast, animal cells and insect cells, and most preferably, it is Escherichia coli.

According to another exemplary embodiment of the present invention, provided is a method for preparing nanoparticles, including the steps of: (a) preparing an expression vector including a polynucleotide encoding a fusion protein, which consists of a ferritin protein; a single-chain variable fragment which is bound to an N-terminus of the ferritin protein; and an N-terminus domain (hLysRS N-terminal appended RNA interacting domain; hRID) which is isolated from a human-derived lysyl tRNA synthetase that is bound to an N-terminus of the single-chain variable fragment; (b) preparing a transformant by introducing the expression vector into a host cell; (c) culturing the transformant to induce expression of the fusion protein; and (d) purifying nanoparticles formed by self-assembly of the expressed fusion protein.

In addition, the present invention provides a pharmaceutical composition for ameliorating or treating a disease, including the nanoparticles. The disease may be specified differently depending on the type of the single-chain variable fragment, and when the single-chain variable fragment is derived from Bevacizumab, cancer (particularly, colon cancer, lung cancer, glioblastoma, renal cell carcinoma) or eye disease (e.g., macular degeneration) may be a disease to be treated (target disease). When the single-chain variable fragment is derived from Trastuzumab, cancer (particularly, breast cancer or metastatic gastric cancer) may be a disease to be treated (target disease). When the single-chain variable fragment is derived from Pertuzumab, the disease to be treated (target disease) may include cancer such as breast cancer, ovarian cancer and ovarian cancer, but the present invention is not limited thereto, but extended to other single-chain variable fragments derived from antibodies for disease therapy.

In addition, the present invention provides a method for preventing or treating a target disease, including the step of administering the self-assembled nanoparticles.

It is preferable to apply the therapeutically effective amount differently according to various factors including the specific composition including the type and degree of the reaction to be achieved, whether other agents are used in some cases, the subject's age, weight, general health status, gender and diet, administration time, administration route and the secretion rate of the composition, the treatment period, drugs used with or concurrently with the specific composition, and similar factors well known in the field of medicine. Therefore, it is preferable to determine the effective amount of the composition suitable for the purpose of the present invention in consideration of the foregoing description.

The subject is applicable to any mammal, and the mammal includes not only humans and primates, but also domestic animals such as cattle, pigs, sheep, horses, dogs and cats.

### Advantageous Effects

The vector for preparing the self-assembled nanoparticles or fusion protein according to the present invention can significantly enhance the water-soluble expression of a single-chain variable fragment-ferritin fusion protein and self-assembled nanoparticles in host cells, and the production efficiency can be increased.

In addition, the fusion protein expressed according to the vector of the present invention forms nanoparticles through self-assembly, and since the formed nanoparticles present a single-chain variable fragment on the surface (surface expression), it has high target affinity, and accordingly, since it increases the efficiency of various immune responses, it can be utilized as a vaccine and therapeutic agent.

### DESCRIPTION OF DRAWINGS

FIG. 1 shows the schematic diagrams of vectors for encoding a fusion protein according to the present invention. FIG. 1a shows a case in which scfv is derived from Bevacizumab, FIG. 1b shows a case in which scfv is derived from Trastuzumab, and

FIG. 2b shows a case in which scfv is derived from Pertuzumab.

FIG. 2 shows the structure of a linker according to the type of scfv and the three-dimensional structure when the linker is combined with scfv according to an exemplary embodiment of the present invention.

FIG. 3 is a set of images confirming the solubility of RID and scfv fusion proteins. FIG. 3a is the results of confirming the water solubility of the hRID-Bevacizumab scfv-ferritin fusion protein, FIG. 3b is the results of confirming the water solubility of the hRID-Trastuzumab scfv-ferritin fusion protein, and FIG. 3c is the results of confirming the water solubility of the hRID-Pertuzumab scfv-ferritin fusion protein.

FIG. 4 is a set of images confirming the isolation of the hRID-Bevacizumab scfv-ferritin fusion protein. In FIG. 4a, the chromatogram results when the hRID-Bevacizumab scfv-ferritin fusion protein eluted by nickel affinity chromatography was purified by ion exchange chromatography was confirmed, and FIG. 4b shows the results of size exclusion chromatography.

FIG. 5 is a set of images confirming the isolation of the hRID-Trastuzumab scfv-ferritin fusion protein. FIG. 5a shows the chromatogram results when the hRID-Trastuzumab scfv-ferritin fusion protein eluted by nickel affinity chromatography was purified through ion exchange chromatography, and FIG. 5b shows the results of size exclusion chromatography.

FIG. 6 is a set of images confirming the isolation of the hRID-Pertuzumab scfv-ferritin fusion protein. FIG. 6a shows the chromatogram results when the hRID-Pertuzumab scfv-ferritin fusion protein eluted by nickel affinity chromatography was purified through ion exchange chromatography, and FIG. 6b shows the results of size exclusion chromatography.

FIG. 7 is a set of transmission electron microscope images of nanoparticles formed by self-assembly of the hRID-Bevacizumab scfv-ferritin fusion protein, hRID-Trastuzumab scfv-ferritin fusion protein and hRID-Pertuzumab scfv-ferritin fusion protein.

FIG. 8 is the results of comparing the stability of Bevacizumab of scFv, hRID-Bevacizumab scfv-ferritin fusion protein nanoparticles and Bevacizumab.

FIG. 9 is a graph comparing the binding affinity of the hRID-Bevacizumab scfv-ferritin (HFH; Human Ferritin heavy chain) fusion protein by ELISA analysis. FIG. 9 shows the folding and binding affinities of hRID(W)-Bevacizumab scfv-ferritin (HFH) fusion protein nanoparticles, hRID(9M)-Bevacizumab scfv-ferritin (HFH) fusion protein nanoparticles and Bevacizumab scfv according to antibody concentration.

FIG. 10 is a graph comparing the binding affinity of hRID-Pertuzumab scfv-ferritin fusion protein nanoparticles to a target by SPR analysis.

FIG. 11 is the results confirming the effect of a multimer of scFv targeting the vascular endothelial growth factor (VEGF) on vascular endothelial cell migration and angiogenesis.

FIG. 12 shows choroidal flat-mount immunofluorescence images obtained 14 days after intravitreal injection of a drug into the eye of a mouse with laser-induced choroidal neovascularization and the average area of the measured choroidal neovascularization.

FIG. 13 is a set of images observing the distribution of scFv in the retina using a confocal microscope saline on day 1 and day 7, after respectively injecting saline, 120 nM ScFv, 600 nM ScFv, 120 nM ScFv-Ferritin and 600 nM ScFv-Ferritin fusion protein nanoparticles into the eyes of mice, respectively.

FIG. 14 shows the stability of fusion protein nanoparticles according to hRID. FIG. 14a shows the hRID-dependent stability of the hRID-Bevacizumab scfv-ferritin fusion protein nanoparticles, FIG. 14b shows the hRID-dependent stability of the hRID-Trastuzumab scfv-ferritin fusion protein nanoparticles, and FIG. 14c shows the hRID-dependent stability of the hRID-Pertuzumab scfv-ferritin fusion protein nanoparticles.

FIG. 15 is a set of images of purified fusion protein nanoparticles including hRID. FIG. 15a is a set of image showing the purification results of hRID-Bevacizumab scfv-ferritin fusion protein nanoparticles when the hRID is wild-type (w) or mutant hRID(9m), respectively, and FIG. 15b shows the results of size exclusion chromatography.

FIG. 16 is a set of images showing the purification results of hRID-Trastuzumab scfv-ferritin fusion protein nanoparticles when the hRID is wild-type (w) or mutant hRID(9m) in the protein, respectively.

FIG. 17 shows TEM images of hRID-Bevacizumab scfv-ferritin fusion protein nanoparticles respectively formed when hRID is wild-type (w) or mutant hRID (9m).

FIG. 18 is a graph comparing the binding affinity of the hRID-Bevacizumab scfv-ferritin (HFL; Human Ferritin light chain) fusion protein by indirect ELISA analysis. FIG. 18 shows the binding affinities of hRID(W)-Bevacizumab scfv-ferritin (HFL) fusion protein nanoparticles, hRID(9M)-Bevacizumab scfv-ferritin (HFL) fusion protein nanoparticles and the folding and targeting of Bevacizumab scfv according to the antibody concentration.

FIG. 19 is a graph analyzing the target binding affinity of hRID-scFv-G3SG3TG3SG3-ferritin (scFv-HFL) nanoparticles, hRID-scFv (scFv) and hRID-HFL (HFL) according to an exemplary embodiment of the present invention by sandwich ELISA. The results were obtained by repeating the ELISA three times.

FIG. 20 is a mimetic diagram showing a structure in which the fusion protein prepared according to an exemplary embodiment of the present invention forms nanoparticles by self-assembly. In FIG. 20, blue represents an antibody heavy chain variable fragment of scfv, and red represents an antibody light chain variable fragment of scfv.

### MODES OF THE INVENTION

Hereinafter, the present invention will be described in detail.

In one aspect, the present invention provides an expression vector for preparing self-assembled nanoparticles, including a polynucleotide encoding a single-chain variable fragment; a peptide for enhancing the expression efficiency of the single-chain variable fragment; and ferritin which is bound to the 3′-end of a polynucleotide encoding the single-chain variable fragment.

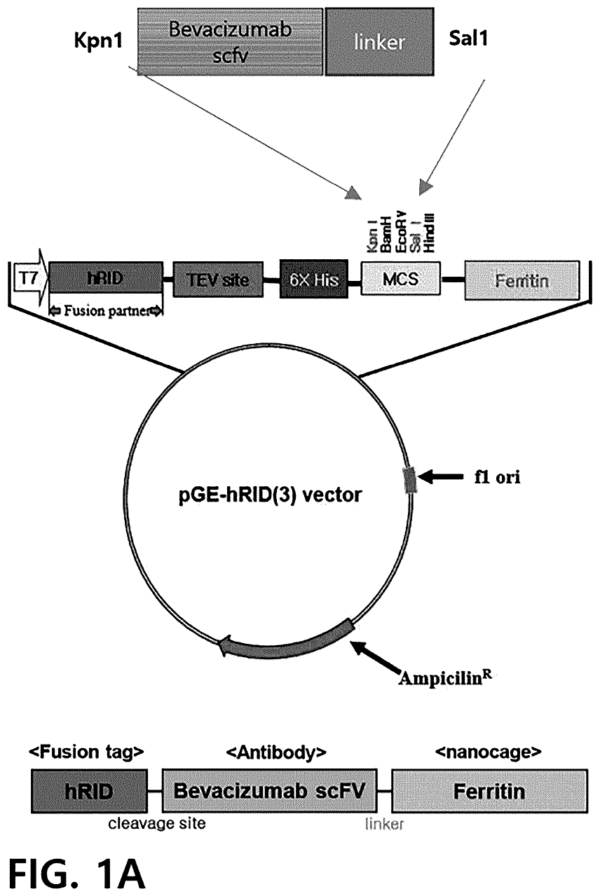

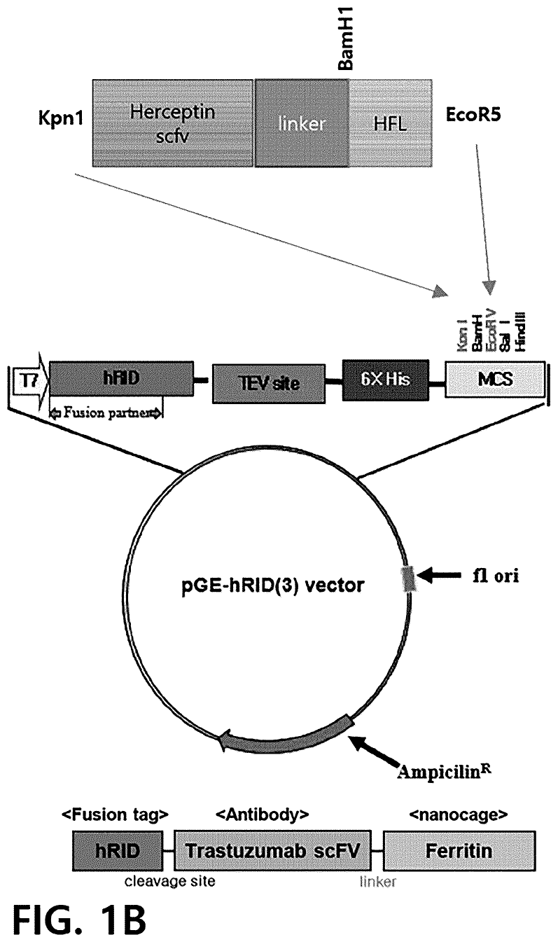

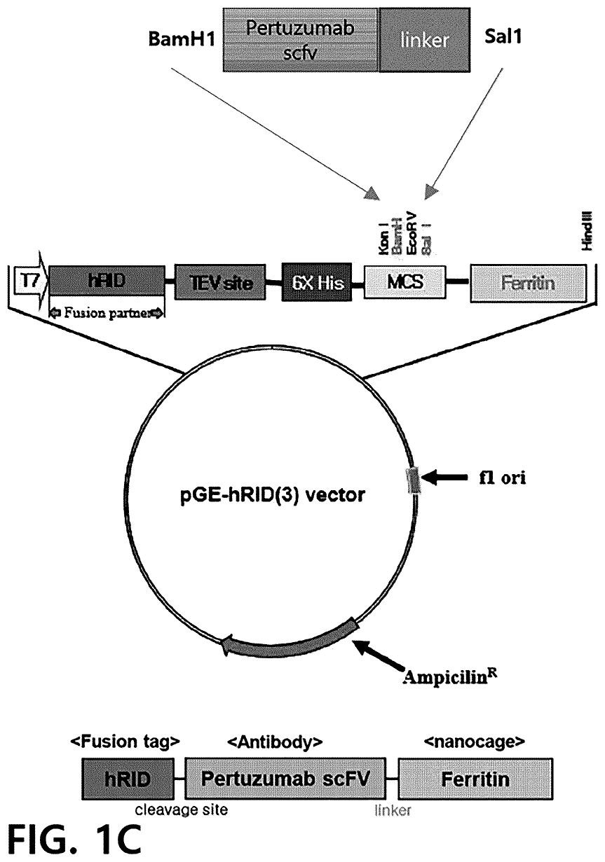

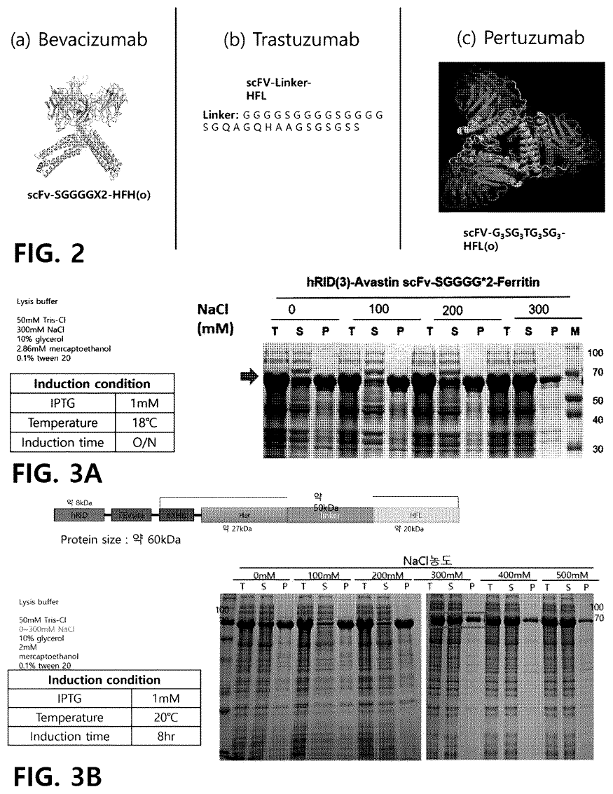

FIG. 1 shows the structures of expression vectors according to the present invention (FIGS. 1a to 1c), wherein hRID is cleaved after promoting the solubility and folding of a single-chain variable fragment-ferritin fusion protein monomer. Thereafter, the monomer may be appropriately folded to form a trimer structure, and 8 trimers may be assembled to form nanoparticles. That is, according to the present invention, the nanoparticles of the present invention may include 1 or more to 24 or less of single-chain variable fragment-ferritin fusion protein monomers, and preferably, the fusion protein monomers may form trimers to prepare nanoparticles including the 8 trimers (a total of 24 fusion protein monomers). On the surface of the nanoparticles, a 24 single-chain variable fragments-ferritin fusion protein may be displayed on the surface.

As used herein, the term “antibody” includes immunoglobulin molecules having antigen-binding ability with reactivity to a specific antigen, and includes both polyclonal and monoclonal antibodies. The term also includes forms produced by genetic engineering, such as chimeric antibodies (e.g., humanized murine antibodies) and heterologous antibodies (e.g., bispecific antibodies). Antibodies are structurally composed of heavy and light chains, each of which includes a constant region and a variable region. The variable regions of the heavy and light chains include three variable regions called “complementarity determining regions (CDRs)” and four “framework regions.”

As used herein, the term “single-chain variable fragment (scFv)” is not a natural fragment formed from an antibody, but is a fusion protein constructed by artificially fusing fragments including the variable region of the heavy chain constituting the antibody and the variable region of the light chain.

In the present invention, the type of the single-chain variable fragment is not limited as long as the nucleic acid or amino acid sequence is known. Preferably, it may be the single-chain variable fragments of Trastuzumab, Bevacizumab and Pertuzumab.

The “single-chain variable fragment” of the present invention is a protein that a person skilled in the art want to produce in large quantities, and it refers to all proteins that can be expressed in a host cell by inserting a polynucleotide encoding the protein into a recombinant expression vector. In the present invention, the “single-chain variable fragment” is expressed in a fusion form with ferritin in a host cell, and it may be displayed on the surface of nanoparticles that are self-assembled by ferritin.

It is reported that the “peptide for enhancing the expression efficiency of a single-chain variable fragment” according to the present invention is a peptide that can express a fusion protein of the single-chain variable fragment and ferritin in a water-soluble form, and for example, Glutathione S transferase (GST), maltose binding protein, ubiquitin, thioredoxin and the like are included. Preferably, it may be selected from fusion proteins, such as rRID (rabbit), mRID (mouse), hRID(human) and LysRS (E. coli), and most preferably, it may be constituted to include the amino acid sequence of an N-terminus domain (hLysRS N-terminus appended RNA interacting domain; hRID) which is separated from a human-derived lysyl tRNA synthetase.

As used herein, the term “RID (RNA interaction domain)” refers to a unique N-terminus extension (about 70 amino acids) involved in the interaction between RNA and other proteins, and it is isolated from human-derived lysyl tRNA synthetase. In a preferred exemplary embodiment of the present invention, tag-RID (represented by SEQ ID NO: 1) in which a tag sequence is bound to RID was used as a binding partner for the soluble expression of single-chain variable fragments (scFv) in bacteria.

In an exemplary embodiment of the present invention, in order to compare whether RNA binding in the RID affects protein folding and nanoparticle structure formation, a double-mutant hRID which was mutated (K19A, K23A) at K19 and K23 sites among 9 hRID domains, and 9 mutant hRIDs which were mutated with K19A, K23A, R24A, K27A, K30A, K31A, K35A, K38A and K40A were created. When using the above two mutant hRID and wild-type hRID(WT) as fusion partners, it was found that the protein folding was improved only when the wild-type hRID(WT) having RNA-binding ability to the hRID was fused such that the nanoparticles were formed well. The mutation sites and amino acid sequences of the mutants are shown in Table 1 below, the amino acid sequence of wild-type tag-hRID(WT) is shown in SEQ ID NO: 1, and the nucleotide sequence is shown in SEQ ID NO: 2.

TABLE 1

| | |
|---|---|
| Tag-hRID(W) amino | MSEQHAQAAVQAAEVKVDGSEPKLSKNEL |
| seq | KRRLKAEKKVAEKEAKQKELSEKQLSATA |
| | AATNHTTDNGVGPEEESV |
| Tag-hRID(2m) amino | MSEQHAQAAVQAAEVKVDGSEPKLSKNEL |
| seq(K19A/K23A) | ARRLAAEKKVAEKEAKQKELSEKQLSATA |
| | AATNHTTDNGVGPEEESV |
| Tag-hRID(9m) amino | MSEQHAQAAVQAAEVKVDGSEPKLSANEL |
| seq(K19A/K23A/R24A/ | AARLAAEAAVAEAEAAQAELSEKQLSATA |
| K27A/K30A/ | AATNHTTDNGVGPEEESV |
| K31A/K35A/K38A/K40A) | |

In the present invention, ferritin may be used without limitation as long as each thereof is a protein that has an activity of being capable of forming a cage-type complex protein as a unit.

In the present invention, the ferritin protein or fragment is not limited as long as the activity of being capable of forming a cage-type complex protein is maintained, and HFL (human ferritin light chain) or HFH (human ferritin heavy chain) may be used. The ferritin protein or fragment thereof according to the present invention may be a protein or fragment formed by partially adding, deleting or substituting amino acid residues in the amino acid sequence of SEQ ID NO: 2.

As used herein, the term “expression vector” is a linear or circular DNA molecule consisting of fragments encoding a polypeptide of interest that is operably linked to additional fragments that serve for transcription of the expression vector. Such additional fragments include promoter and terminator sequences. The expression vector also includes one or more origins of replication, one or more selectable markers, polyadenylation signals and the like. Expression vectors are generally derived from plasmid or viral DNA, or contain elements of both.

As used herein, the term “operably linked” refers to the arrangement of fragments in a promoter to act to initiate transcription and progress through the coding sequence to the termination codon.

In the expression vector according to the present invention, the expression vector may be a plasmid, a viral vector, a phage particle or a genome insert. After the expression vector is transformed into a host cell, it may be cloned independently of the genome of a host cell or integrated into the genome of a host cell.

In an exemplary embodiment of the present invention, the vector may be constructed by further including a linker between the polynucleotide encoding a single-chain variable fragment and ferritin.

The linker is for attaching the single-chain variable fragment (scFv) to a specific site at the C-terminus or N-terminus of the ferritin monomer fragment, and it may consist of one to several amino acids.

In the present invention, the linker is introduced between the single-chain variable fragment and ferritin to minimize steric hindrance between the two domains, thereby improving the formation of trimers and nanoparticle structures.

In the present invention, the nanoparticles are formed by the precise self-assembly properties of low-molecular-weight monomers, and refer to cage-type particles made of proteins having a space therein. The nanoparticles of the present invention are characterized in that they include the single-chain variable fragment (scFv)-ferritin fusion protein of the present invention as a monomer constituting the cage.

As used herein, the term “self-assembly” refers to the property of certain molecules to form specific nanostructures on their own without a special external stimulus or artificial induction.

In the nanoparticles of the present invention, after the single-chain variable fragment-ferritin fusion protein monomer fragment forms a trimer, the 8 trimers are regularly arranged three-dimensionally, and this may be formed by gathering 24 monomers.

Meanwhile, according to the present invention, a nucleic acid encoding the single-chain variable fragment-ferritin fusion protein or a recombinant vector including the nucleic acid may transform or transfect a host cell.

As used herein, the term “nucleic acid” or “polynucleotide” refers to deoxyribonucleotides or ribonucleotides in single or double-stranded form. Unless otherwise limited, it also includes known analogs of natural nucleotides that hybridize to nucleic acids in a manner similar to naturally occurring nucleotides.

As used herein, the term “recombinant protein” or “fusion protein” refers to a protein in which another protein is linked to the N-terminus or C-terminus of the single-chain antibody fragment (scFv) sequence or a different amino acid sequence is added thereto, and preferably, it refers to a monomeric protein (scFv-ferritin monomer) in which the single-chain antibody fragment (scFv) is bound to the N-terminus of the ferritin protein, or a monomeric protein (hRID-scFv-ferritin monomer) in which the single-chain antibody fragment (scFv) is bound to the N-terminus of the ferritin protein and hRID is bound to the single-chain antibody fragment.

As used herein, the term “protein” is used interchangeably with “peptide” or “polypeptide”, and for example, it refers to a polymer of amino acid residues as commonly found in naturally occurring proteins.

The recombinant vector of the present invention may be obtained by linking (inserting) the nucleic acid of the present invention to an appropriate vector. The vector into which the nucleic acid of the present invention is inserted is not particularly limited as long as it can be replicated in a host. For example, plasmid DNA, phage DNA and the like may be used. Specific examples of the plasmid DNA include commercial plasmids such as pCDNA31+ (Invitrogen). Other examples of plasmids that can be used in the present invention include E. coli-derived plasmids (pYG601BR322, pBR325, pUC118 and pUC119), Bacillus subtilis-derived plasmids (pUB110 and pTP5) and yeast-derived plasmids (YEp13, YEp24 and YCp50). Specific examples of phage DNA include λ-phages (Charon4A, Charon21A, EMBL3, EMBL4, λgt10, λgt11 and λZAP). In addition, animal viruses such as retroviruses, adenoviruses or vaccinia viruses, and insect viruses such as baculoviruses may also be used.

In order to insert the nucleic acid of the present invention into a vector, the method of cleaving the purified DNA with an appropriate restriction enzyme and inserting the same into an appropriate restriction site or cloning site of the vector DNA may be used.

Preferably, the nucleic acid of the present invention is operably linked to a vector. In addition to the promoter and the nucleic acid of the present invention, the vector of the present invention may further include a cis element such as an enhancer, a splicing signal, a poly A addition signal, a selection marker, a ribosome binding sequence (SD sequence) and the like. As examples of the selection marker, chloramphenicol-resistant nucleic acid, ampicillin-resistant nucleic acid, dihydrofolate reductase, neomycin-resistant nucleic acid and the like may be used, but the additional components that are operably linked are not limited to the above examples.

In another aspect, the present invention provides a host cell which is transformed by the above-described expression vector.

As used herein, the term ‘transformation’ refers to introducing DNA into a host such that DNA can be replicated as a factor of chromosomes or by chromosomal integration completion, and it refers to a phenomenon of artificially causing genetic changes by introducing external DNA into cells.

Any transformation method may be used for the transformation method of the present invention, and it can be easily performed according to a conventional method in the art. In general, examples of the transformation method include CaCl₂) precipitation, the Hanahan method which increases efficiency by using a reducing substance called DMSO (dimethyl sulfoxide) in the CaCl₂) method, electroporation, calcium phosphate precipitation, protoplast fusion, agitation using silicon carbide fibers, agrobacteria-mediated transformation, transformation using PEG, dextran sulfate, lipofectamine and drying/inhibition-mediated transformation method

The method for transforming a nucleic acid encoding the single-chain variable fragment-ferritin fusion protein or a vector including the same according to the present invention is not limited to the above examples, and transformation or transfection methods that are commonly used in the art may be used without limitation.

The transformant of the present invention may be obtained by introducing a nucleic acid encoding the single-chain variable fragment-ferritin fusion protein, which is a target nucleic acid, or a recombinant vector including the same into a host.

The host is not particularly limited as long as it allows expression of the nucleic acid of the present invention. Specific examples of the host that can be used in the present invention include prokaryotic cells, such Escherichia bacteria such as E coli; Bacillus bacteria such as Bacillus subtilis; Pseudomonas bacteria such as Pseudomonas putida; lactic acid bacteria such as Lactobacillus and Enterococcus, yeasts such as Saccharomyces cerevisiae and Schizosaccharomyces pombe; animal cells and insect cells.

In previous studies, eukaryotic cells such as yeast, insect cells and mammalian cells have been preferred over E. coli due to auxiliary folding, post-translational modification and the possibility of generating multi-component nanoparticles, but when these eukaryotic cells are used, there is a problem in that the mass production process is difficult.

In an exemplary embodiment of the present invention, in order to solve the above problems, a self-assembled nanoparticle structure which is marked with a single-chain variable fragment was produced by using E. coli, which is inexpensive and can be easily mass-produced.

Accordingly, the host cell of the present invention is not limited thereto, but it may be E. coli in terms of inexpensive and easy mass production.

When a bacterium such as E. coli is used as a host, the recombinant vector of the present invention is capable of autonomous replication in the host, and it is composed of a promoter, a ribosome binding sequence, the nucleic acid of the present invention and a transcription termination sequence.

As the promoter of the present invention, any promoter may be used as long as the nucleic acid of the present invention is expressed in a host such as E. coli. For example, E. coli- or phage-derived promoters such as trp promoter, lac promoter, PL promoter or PR promoter; E. coli infected phage-derived promoters such as T7 promoter may be used. In addition, an artificially modified promoter such as tac promoter may also be used.

In another aspect, the present invention provides a method for preparing self-assembled nanoparticles, including the steps of (a) preparing an expression vector a polynucleotide encoding a single-chain variable fragment; a peptide for enhancing the expression efficiency of the single-chain variable fragment; and a polynucleotide encoding ferritin; (b) preparing a transformant by introducing the expression vector into a host cell; (c) culturing the transformant to induce the expression of a single-chain variable fragment-ferritin fusion protein; and (d) purifying nanoparticles formed by self-assembly of the expressed single-chain variable fragment-ferritin fusion protein.

Specific details of the expression vector, transformant and self-assembled nanoparticles are the same as described above.

In order to facilitate the purification of the single-chain variable fragment recovered in the present invention, the plasmid vector may further include other sequences if necessary. The sequences that may be further included may be a tag sequence for protein purification, and for example, glutathione S-transferase (Pharmacia, USA), MBP (maltose binding protein, USA), FLAG (IBI, USA) and hexahistidine (Quiagen, USA), and most preferably, it may be hexahistidine, but the types of sequences required for the purification of the single-chain variable fragment are not limited by the above examples.

In the case of a fusion protein expressed by a vector including the fusion sequence, it may be purified by affinity chromatography. For example, when glutathione-S-transferase is fused, glutathione, which is a substrate of this enzyme, may be used, and when MBP is used, the desired single-chain variable fragment (scFv) may be easily recovered by using an amylose column.

In order to express the single-chain variable fragment-ferritin fusion protein of the present invention, the host cell transformed by the above method may be cultured by conventional methods used in the art. For example, the transformant expressing the single-chain variable fragment-ferritin fusion protein may be cultured in various media, and fed-batch culture and continuous culture may be performed, but the method for culturing the transformant of the present invention is not limited by the above examples. In addition, the carbon source that can be included in the medium for the growth of host cells may be appropriately selected by the judgment of a person skilled in the art according to the type of a prepared transformant, and appropriate culture conditions may be adopted to control the culture period and amount.

When an appropriate host cell is selected and the medium conditions are established, the transformant which is successfully transformed with a single-chain variable fragment produces the single-chain variable fragment-ferritin fusion protein, and the single-chain variable fragment-ferritin fusion protein produced according to the composition of the vector and the characteristics of the host cell may be produced in the cytoplasm of the host cell, and secreted into the periplasmic space or the extracellular space.

Proteins expressed in or outside the host cell may be purified in a conventional manner. As examples of purification methods, the protein of the present invention may be purified by applying techniques such as salting out (e.g., ammonium sulfate precipitation, sodium phosphate precipitation, etc.), solvent precipitation (e.g., protein fraction precipitation using acetone, ethanol, etc.), dialysis, gel filtration, ion exchange, chromatography such as reversed-phase column chromatography and ultrafiltration alone or in combination.

In another aspect, the present invention provides nanoparticles prepared by the above preparation method. The single-chain variable fragment-ferritin fusion protein according to the present invention may form spherical nanoparticles by self-assembly of 24 monomers. In this case, the diameter of the nanoparticles may be 24 to 30 nm.

Specific details of the nanoparticles are the same as those described above.

In another aspect, the present invention provides a pharmaceutical composition for preventing, ameliorating or treating a disease, including the nanoparticles.

In the present invention, “prevention” refers to any action that suppresses or delays the onset of a target disease by administration of the composition, and “treatment” refers to any action in which the symptoms of the target disease are improved or beneficially changed by the composition.

In the present invention, the “disease” may be specified differently depending on the type of a single-chain variable fragment used. For example, when the single-chain variable fragment is derived from Bevacizumab, cancer (particularly, colon cancer, lung cancer, glioblastoma, renal cell cancer) or eye disease (e.g., macular lesion) may be the target disease to be treated. When the single-chain variable fragment is derived from Trastuzumab, cancer (particularly, breast cancer or metastatic gastric cancer) may be a target disease. When the single-chain variable fragment is derived from Pertuzumab, cancers such as breast cancer, ovarian cancer and ovarian cancer may be included as target diseases.

The pharmaceutical composition according to the present invention may contain the nanoparticles alone or may be formulated in a suitable form together with a pharmaceutically acceptable carrier, and may further contain excipients or diluents. As used herein, the term ‘pharmaceutically acceptable’ refers to a non-toxic composition that is physiologically acceptable and does not normally cause allergic reactions such as gastrointestinal disorders, dizziness or similar reactions when administered to humans.

The pharmaceutically acceptable carrier may further include, for example, a carrier for oral administration or a carrier for parenteral administration.

The carriers for oral administration may include lactose, starch, cellulose derivatives, magnesium stearate, stearic acid and the like.

Moreover, various drug delivery materials used for oral administration to a peptide agent may be included. In addition, the carriers for parenteral administration may include water, suitable oils, saline, aqueous glucose and glycols, and further include stabilizers and preservatives. Suitable stabilizers include antioxidants such as sodium hydrogen sulfite, sodium sulfite or ascorbic acid. Suitable preservatives include benzalkonium chloride, methyl- or propyl-parabens and chlorobutanol.

The pharmaceutical composition of the present invention may further include a lubricant, a humectant, a sweetener, a flavoring agent, an emulsifier and a suspension agent in addition to the above components. For other pharmaceutically acceptable carriers and agents, reference may be made to those described in the following literature (Remington's Pharmaceutical Sciences, 19th ed, Mack Publishing Company, Easton, PA, 1995).

The composition of the present invention may be administered to mammals including humans by any method. For example, it may be administered orally or parenterally. Parenteral administration methods include intravenous, intramuscular, intraarterial, intramedullary, intrathecal, intracardiac, transdermal, subcutaneous, intraperitoneal, intranasal, enteral, topical, sublingual or rectal administration, but the present invention is not limited thereto.

The pharmaceutical composition of the present invention may be formulated into a preparation for oral or parenteral administration according to the route of administration as described above.

In the case of a formulation for oral administration, the composition of the present invention may be formulated using a method known in the art as a powder, granule, tablet, pill, dragee, capsule, liquid, gel, syrup, slurry, suspension and the like. For example, as oral preparations, tablets or dragees may be obtained by mixing the active ingredient with a solid excipient, pulverizing the same, adding a suitable auxiliary agent and processing into a granule mixture. Examples of excipients include sugars including lactose, dextrose, sucrose, sorbitol, mannitol, xylitol, erythritol and maltitol, starches including corn starch, wheat starch, rice starch and potato starch, celluloses including cellulose, methyl cellulose, sodium carboxymethylcellulose, and hydroxy propylmethyl-cellulose, and fillers such as gelatin, polyvinylpyrrolidone and like. In addition, in some cases, cross-linked polyvinylpyrrolidone, agar, alginic acid or sodium alginate may be added as a disintegrant.

Furthermore, the pharmaceutical composition of the present invention may further include an anti-aggregating agent, a lubricant, a wetting agent, a fragrance, an emulsifying agent and a preservative. In the case of a formulation for parenteral administration, it may be formulated in the form of injections, creams, lotions, ointments for external use, oils, moisturizers, gels, aerosols and nasal inhalants by a method known in the art. These formulations are described in the literature (Remington's Pharmaceutical Science, 19th ed, Mack Publishing Company, Easton, PA, 1995), which is a generally known prescription for all pharmaceutical chemistry.

The total effective amount of the pharmaceutical composition of the present invention may be administered to a patient in a single dose, and may be administered by a fractionated treatment protocol with multiple doses for a long period of time. The pharmaceutical composition of the present invention may vary the content of the active ingredient depending on the degree of symptoms of the disease. Preferably, the preferred total dosage of the pharmaceutical composition of the present invention may be about 0.01 μg to 10,000 mg per kg of patient body weight per day, and most preferably, 0.1 mg to 500 mg per kg of patient body weight per day. However, since the dosage of the pharmaceutical composition of the present invention may be determined in consideration of various factors such as age, weight, health condition, gender, disease severity, diet, excretion rate and the like, one of ordinary skill in the art will be able to determine the appropriate effective dosage of the composition of the present invention in consideration of the above points. The pharmaceutical composition according to the present invention is not particularly limited in its formulation, route of administration and method of administration as long as it shows the effects of the present invention.

Hereinafter, various examples are presented to help the understanding of the invention. The following examples are provided for easier understanding of the invention, and the protection scope of the invention is not limited to the following examples.

### Experimental Method

### 1. Construction of Protein Expression Vectors

1-1. Construction of Fusion Protein Vector Using Single-Chain Variable Fragment Derived from Bevacizumab (FIG. 1a)

After enzymatic digestion of the LysRS gene with nde1 and kpn1 in the pGE-LysRS vector, the PCR product of hRID(RNA interaction domain originated from human), which is 1 to 69 amino acids at the N-terminus of human LysRS, was excised using the same restriction enzymes to create a pGE-hRID vector. Next, the pGE-hRID vector was digested with SalI and Hind² restriction enzymes, and a ferritin gene derived from a microorganism or human was inserted to create a hRID-FR vector. The hRID-FR vector was digested with KpnI and SalI restriction enzymes, and the amino acid SGGGGSGGGG was inserted as a linker between the ScFv sequence ferritin of Bevacizumab to construct a vector composed of a TEV cleavage site and 6x-His-Bevacizumab ScFv-FR in hRID.

1-2. Construction of Fusion Protein Vector Using Single-Chain Variable Fragment Derived from Trastuzumab (FIG. 1b)

The pGE-hRID vector was digested with KpnI and EcoRV restriction enzymes, and GGGG SGGGGSGGGGSGQAGQHAAGSGSGSS was inserted as a linker between the ScFv of Trastuzumab and the human-derived ferritin gene to construct a vector composed of a TEV cleavage site and 6x-His-Trastuzumab ScFv-FR in hRID.

1-3. Construction of Fusion Protein Vector Using Single-Chain Variable Fragment Derived from Pertuzumab (FIG. 1c)

The hRID-FR vector was digested with BamHI and SalI restriction enzymes, and GGGSGGGTGGGSGGG was inserted as a linker between the ScFv sequence of Pertuzumab and Ferritin to construct a vector composed of a TEV cleavage site and 6x-His-Trastuzumab ScFv-FR in hRID.

### 2. Confirmation of Enhancement of Water Solubility

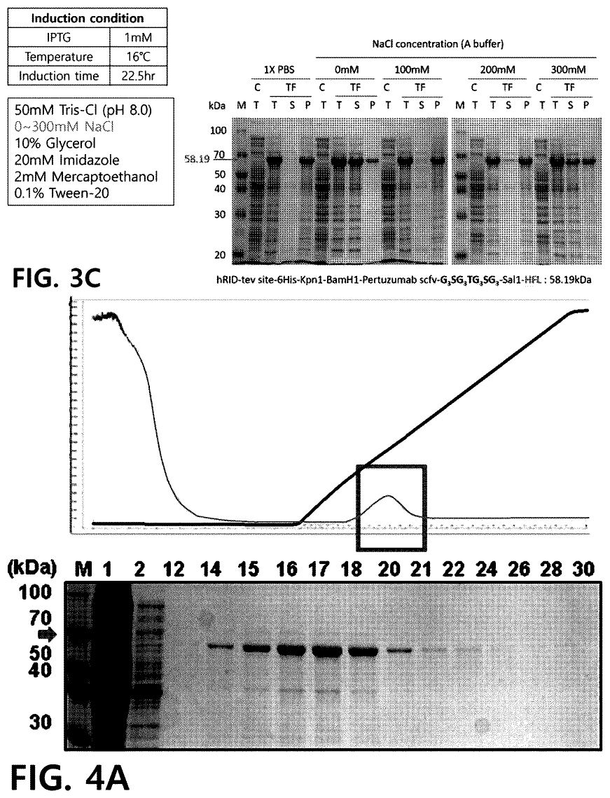

### 2-1. Bevacizumab-Ferritin Fusion Protein Nanoparticles (FIG. 3a)

All plasmids were transformed into SHuffle® T7 Competent E. coli. After treatment with 50 μg/mL of ampicillin in 3 mL of LB medium, it was cultured overnight at 30° C. Each transformant was scaled up in 15 mL of LB medium containing ampicillin, and after the induction of 1 mM IPTG (isopropyl β-D-1-thioglalactopyranoside) at 18° C. and O.D=0.6 to 0.8, the protein was expressed overnight. E. coli was centrifuged to settle the cells, and the cells were disrupted by using ultrasound in wash buffer. In this case, the wash buffer with [50 mM Tris-HCl (pH 8.0), 0, 100, 200, 300 mM NaCl, 10% glycerol, 2.86 mM 2-mercaptoethanol, TWEEN-20 0.1%] was used, and all of the disrupted cells were classified as T (Total), the supernatant of the disrupted cells centrifuged at 12,000 rpm for 20 minutes at 4° C. was classified as S (Soluble), and the settled portion were classified as P (Pellet) to confirm the water solubility of the protein obtained from the disrupted cells by running on SDS PAGE gel.

### 2-2. Trastuzumab-Ferritin Fusion Protein Nanoparticles (FIG. 3b)

All plasmids were transformed into SHuffle® T7 Competent E. coli. After treatment with 50 μg/mL of ampicillin in 3 mL of LB medium, it was cultured overnight at 30° C. Each transformant was scaled up in 15 mL of LB medium containing ampicillin, and after the induction of 1 mM IPTG (isopropyl β-D-1-thioglalactopyranoside) at 20° C. and O.D=0.6 to 0.8, the protein was expressed for 8 hours. E. coli was centrifuged to settle the cells, and the cells were disrupted by using ultrasound in wash buffer. In this case, the wash buffer with [50 mM Tris-HCl (pH 8.0), 0, 100, 200, 300 mM NaCl, 10% glycerol, 2.86 mM 2-mercaptoethanol, TWEEN-20 0.1%] was used, and all of the disrupted cells were classified as T (Total), the supernatant of the disrupted cells centrifuged at 12,000 rpm for 20 minutes at 4° C. was classified as S (Soluble), and the settled portion were classified as P (Pellet) to confirm the water solubility of the protein obtained from the disrupted cells by running on SDS PAGE gel.

### 2-3. Pertuzumab-Ferritin Fusion Protein Nanoparticles (FIG. 3c)

All plasmids were transformed into SHuffle® T7 Competent E. coli. After treatment with 50 μg/mL of ampicillin in 3 mL of LB medium, it was cultured overnight at 30° C. Each transformant was scaled up in 15 mL of LB medium containing ampicillin, and after the induction of 1 mM IPTG (isopropyl β-D-1-thioglalactopyranoside) at 16° C. and O.D=0.6 to 0.8, the protein was expressed for 22.5 hours. E. coli was centrifuged to settle the cells, and the cells were disrupted by using ultrasound in wash buffer. In this case, the wash buffer with [50 mM Tris-HCl (pH 8.0), 0, 100, 200, 300 mM NaCl, 10% glycerol, 2 mM 2-mercaptoethanol, TWEEN-20 0.1%] was used, and all of the disrupted cells were classified as T (Total), the supernatant of the disrupted cells centrifuged at 12,000 rpm for 20 minutes at 4° C. was classified as S (Soluble), and the settled portion were classified as P (Pellet) to confirm the water solubility of the protein obtained from the disrupted cells by running on SDS PAGE gel.

### 3. Protein Expression and Purification (Purification: Ni-Affinity Chromatography and Size Exclusion Chromatography (SEC))

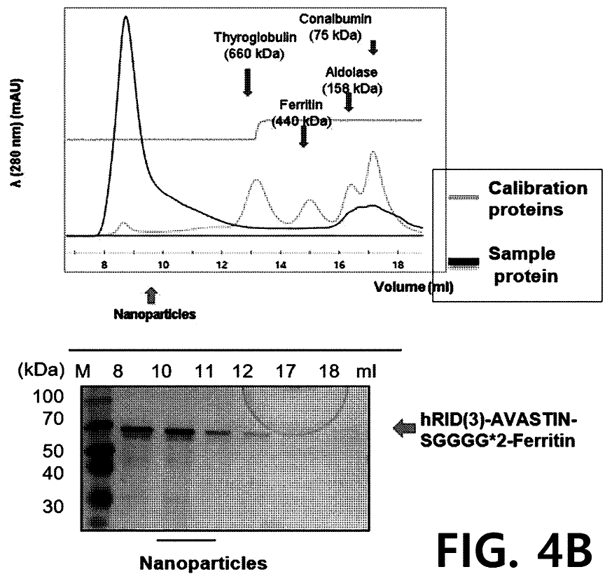

### 3-1. Purification of Bevacizumab-Ferritin Fusion Protein Nanoparticles (FIG. 4a, b)

All plasmids were transformed into SHuffle® T7 Competent E. coli. After treatment with 50 μg/mL of ampicillin in 50 mL of LB medium, it was cultured at 30° C. overnight. Each transformant was cultured in a large volume at 18° C. in 500 mL of LB medium containing ampicillin, and after the induction of 1 mM IPTG (isopropyl β-D-1-thioglalactopyranoside) at O.D=0.6 to 0.8, the protein was expressed overnight. E. coli was centrifuged to settle the cells, and the cells were disrupted by using ultrasound in wash buffer (0 mM NaCl). For protein purification, Histrap HP column (GE healthcare, 17-5248-02) was used, and the wash buffer with [50 mM Tris-HCl (pH 8.0), 300 mM NaCl, 10% glycerol, 2.86 mM 2-mercaptoethanol, TWEEN-20 0.1%, and 20 mM imidazole] was used, and the elution buffer with [50 mM Tris-HCl (pH 8.0), 300 mM NaCl, 10% glycerol, 2.86 mM 2-mercaptoethanol, 0.1% TWEEN-20, and 300 mM imidazole] was used to purify and isolate the protein (Affinity Chromatography). After concentration with centrifugal filters (Centriprep, Merck Millipore Ltd), nanoparticles were purified (SEC) by using Superose 6 10/300 column (GE Healthcare).

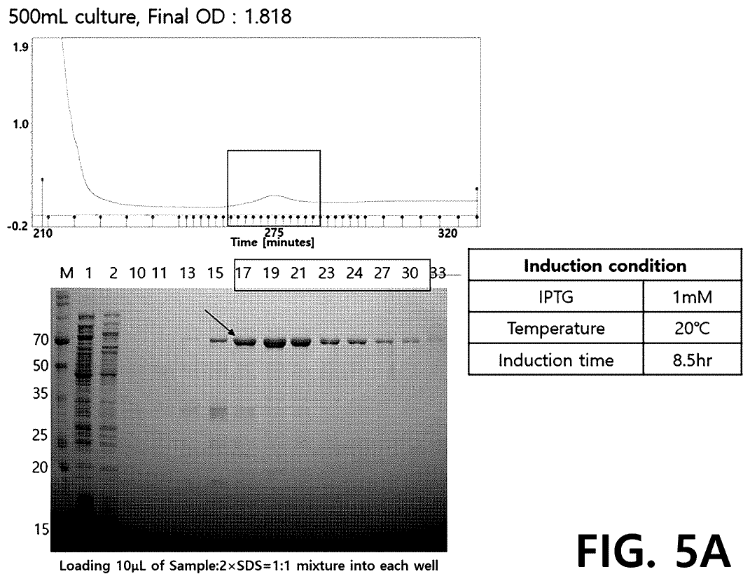

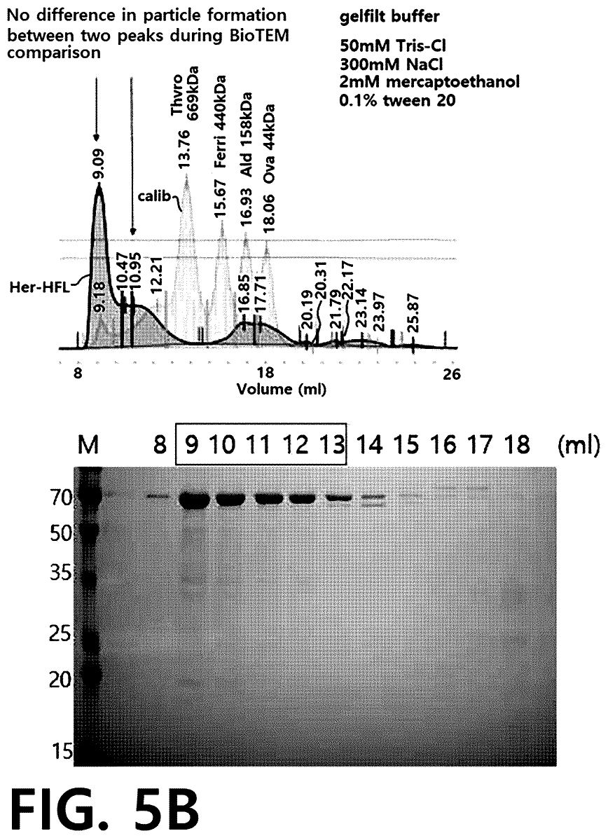

### 3-2. Purification of Trastuzumab-Ferritin Fusion Protein Nanoparticles (FIG. 5a, b)

All plasmids were transformed into SHuffle® T7 Competent E. coli. After treatment with 50 μg/mL of ampicillin in 50 mL of LB medium, it was cultured at 30° C. overnight. Each transformant was cultured in a large volume in 500 mL of LB medium containing ampicillin at 20° C., and after the induction of 1 mM IPTG (isopropyl β-D-1-thioglalactopyranoside) O.D=0.6 to 0.8, the protein was expressed for 8 hours. E. coli was centrifuged to settle the cells, and the cells were disrupted by using ultrasound in wash buffer (0 mM NaCl). For protein purification, Histrap HP column (GE healthcare, 17-5248-02) was used, and the wash buffer with [50 mM Tris-HCl (pH 8.0), 300 mM NaCl, 10% glycerol, 2.86 mM 2-mercaptoethanol, TWEEN-20 0.1%, and 20 mM imidazole] was used, and the elution buffer with [50 mM Tris-HCl (pH 8.0), 300 mM NaCl, 10% glycerol, 2.86 mM 2-mercaptoethanol, 0.1% TWEEN-20, and 300 mM imidazole] was used to purify and isolate the protein (Affinity Chromatography). After concentration with centrifugal filters (Centriprep, Merck Millipore Ltd), nanoparticles were purified (SEC) by using Superose 6 10/300 column (GE Healthcare).

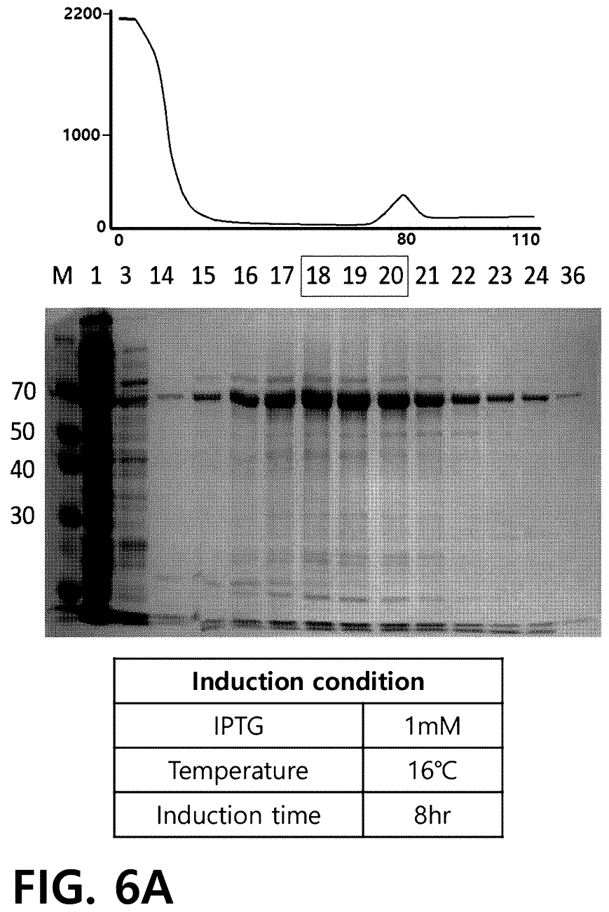

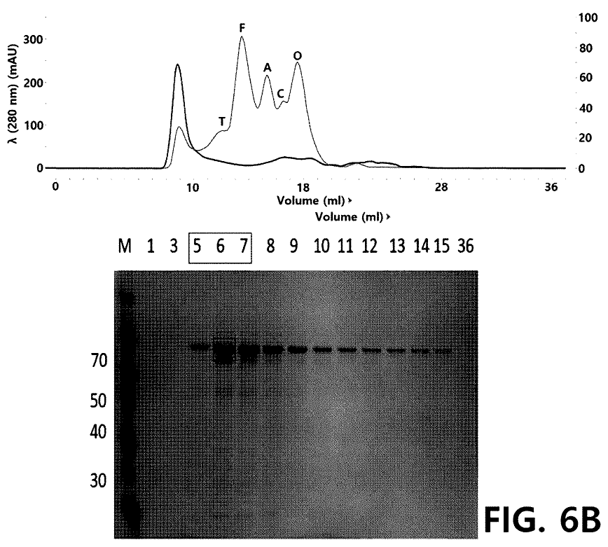

### 3-3. Purification of Pertuzumab-Ferritin Fusion Protein Nanoparticles (FIGS. 6a, b)

All plasmids were transformed into SHuffle® T7 Competent E. coli. After treatment with 50 μg/mL of ampicillin in 50 mL of LB medium, it was cultured at 30° C. overnight. Each transformant was cultured in a large volume in 500 mL of LB medium containing ampicillin at 16° C., and after the induction of 1 mM IPTG (isopropyl β-D-1-thioglalactopyranoside) at O.D=0.6 to 0.8, the protein was expressed for 8 hours. E. coli was centrifuged to settle the cells, and the cells were disrupted by using ultrasound in wash buffer (0 mM NaCl). For protein purification, Histrap HP column (GE healthcare, 17-5248-02) was used, and the wash buffer with [50 mM Tris-HCl (pH 8.0), 300 mM NaCl, 10% glycerol, 2 mM 2-mercaptoethanol, TWEEN-20 0.1%, and 20 mM imidazole] was used, and the elution buffer with [50 mM Tris-HCl (pH 8.0), 300 mM NaCl, 10% glycerol, 2.86 mM 2-mercaptoethanol, 0.1% TWEEN-20, and 300 mM imidazole] was used to purify and isolate the protein (Affinity Chromatography). After concentration with centrifugal filters (Centriprep, Merck Millipore Ltd), nanoparticles were purified (SEC) by using Superose 6 10/300 column (GE Healthcare).

### 4. Assembly of Nanoparticles

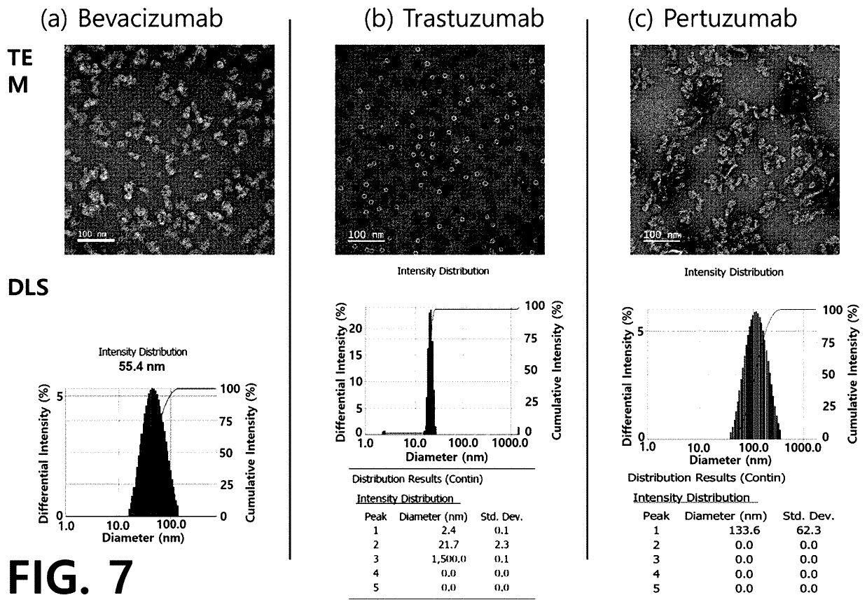

KOREA BASIC SCIENCE INSTITUTE CHUNCHEON CENTER was commissioned to confirm the formation of nanoparticles through bio-TEM. The result is shown in FIG. 7.

Nanoparticle protein (3 mL) was placed in Dispo-H cell, and the size was measured and analyzed by Dynamic Light Scattering (DLS) by using Zeta-potential & Particle size Analyzer (ELS-2000ZS; Otsuka Electronics). The intensity distribution diameter of nanoparticles was measured twice in a water solvent at 25° C., and the sample accumulation time was measured at 200 seconds.

### 5. Stability of Nanoparticles

FIG. 8 is a diagram of the thermal stability analysis of a control material and nanoparticles, and FIG. 5 is the results of measuring signals at a wavelength of 216 nm while increasing the temperature to 19.68 to 98.18° C. of the control material scFv, Bevacizumab and h-scFV-HFH (of nanoparticles) by using circular dichroism (CD) spectroscopy. In this case, in h-scFV-HFH (nanoparticle) and Bevacizumab (control antibody), no noticeable structural change was observed below the temperature of Tm=61.81° C., but the structural change was observed in scFv at 45.71° C. This result shows that our h-scFV-HFH (nanoparticle) has thermal stability that is similar to that of a monoclonal antibody (control material) and more excellent thermal stability than scFv.

### EXAMPLE

### Example 1. SPR and ELISA Analysis

### Example 1-1. SPR and ELISA Analysis of Bevacizumab-Ferritin Fusion Protein SPR Analysis

TABLE 2
Bevacizumab SPR Assay

| Antigen | Antibody | Ka (1/Ms) | Kd (1/s) | KD (M) |
|---|---|---|---|---|
| Human | Bevacizumab | 1.786E+5 | 4.021E−6 | 2.251E−11 |
| VEGF | scFv monomer | 4.574E+4 | 5.682E−4 | 1.242E−8 |
| | h(3)-scFv-HFH | 4.655E+5 | 4.424E−5 | 9.503E−11 |
| | h(3)-scFv-HFH + | 3.813E+4 | 2.577E−6 | 1.241E−11 |
| | chaperone | | | |
| Mouse | Bevacizumab | ND | ND | ND |
| VEGF | scFv monomer | 4.859E+4 | 8.971E−4 | 1.846E−8 |
| | h(3)-scFv-HFH | 7.441E+4 | 3.686E−6 | 4.953E−11 |
| | h(3)-scFv-HFH + | 9.727E+4 | 1.248E−6 | 1.283E−11 |
| | chaperone | | | |

The binding force between Bevacizumab and VEGF, which is the prototype of scFv, and the binding force between the scFv-ferritin fusion protein nanoparticles and VEGF were compared by using SPR (Surface plasmon resonance) analysis.

Specifically, a Biacore T100 analyzer (GE Healthcare, Uppsala, Sweden) was used, and in the biosensor analysis, VEGF protein (10 μg/mL in 10 mM Sodium acetate, pH 5.0) was immobilized by flowing to a CM5 sensor chip (GE Healthcare, Uppsala, Sweden), which was activated by EDC/NHS (N′-(3-dimethylaminopropyl) carbodiimide hydrochloride/N-hydroxysuccinimide), for 5 minutes at a flow rate of 10 μL/min. The activated portion remaining on the surface of the sensor chip was deactivated by adding 1.0 M ethanolamine (pH 8.5). In this state, after Bevacizumab or scFv-ferritin fusion protein was diluted at each concentration in a buffer solution (1×PBS, pH 7.4), the binding sensorgram and dissociation sensorgram were determined while flowing at a flow rate of 30 μL/min. In this case, in order to correct sensorgrams caused by non-specific binding to the sensor chip, cells that were completely inactivated only by BSA (bovine serum albumin) were used, and non-specific binding was corrected by subtracting the BSA sensorgram from the VEGF sensorgram. The surface of the sensor chip was regenerated by flowing a regeneration buffer solution (20 mM NaOH).

### ELISA Analysis

In order to confirm the RNA-mediated folding and stability of hRID as a fusion partner, Bevacizumab-ferritin fusion protein, which is hRID(9m) that cannot bind RNA due to a mutation in the RNA binding site of hRID, was performed to determine whether the binding affinity of hRID was reduced compared to the Bevacizumab-ferritin fusion protein without mutation. 2,000 nM/well hVEGF protein (Abcam) was coated on Nunc 96-well microtiter immunoplates (Thermo Fisher Scientific) overnight at 4° C. Between all processes, immunoplates were washed three times with PBST (0.05% Tween 20 added to PBS) Wash buffer. Then, it was reacted with blocking buffer (5% skim milk added to PBST) at 37° C. for 1 hour. hRID(W)-scFV-HFH, hRID(9m)-scFv-HFH and scFv were serially diluted in ½ from 100 nM to 0.19 nM by using PBST. The diluted materials were applied to immunoplates and incubated for 2 hours. Afterwards, 100 μL of 1/400 anti-hVEGF mAb was added to each well at 37° C. and incubated for 2 hours. Then, peroxidase-conjugated anti-mouse IgG that was diluted by 1/20,000 in serum dilution buffer (PBST+0.5% BSA) was added to the wells and incubated for 2 hours. Finally, 100 μL/well of substrate TMB solution (BD Biosciences) was incubated at 37° C. for 30 minutes in the dark. In order to stop color reaction, after adding 50 μL of stop solution (2NH₂SO₄) to the well, absorbance was measured at 450 μM by using an ELISA reader FLUOstar OPTIMA (BMG LABTECH).

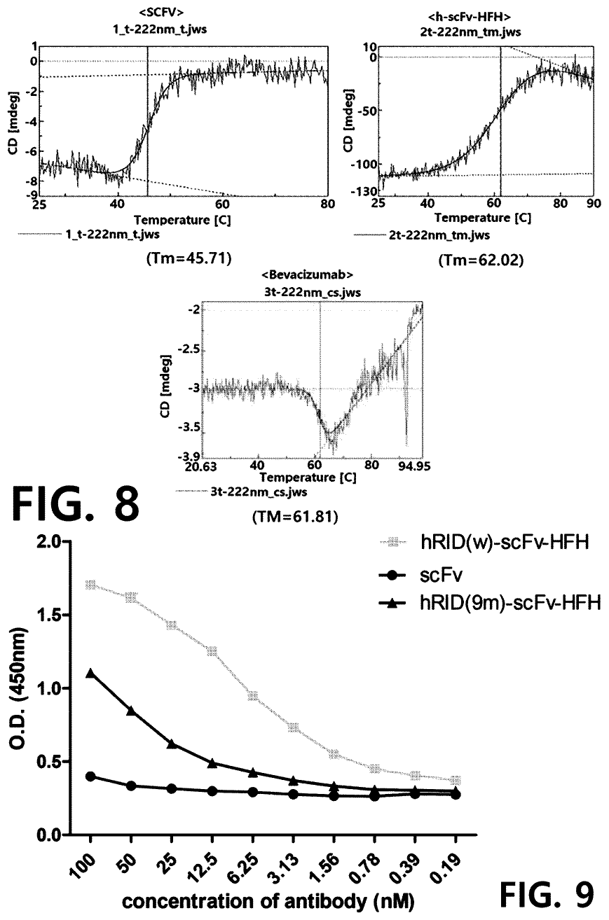

FIG. 9 is the results of confirming the effect of RNA on protein folding through hRID mutation, and it is the results of confirming that the binding force of the Bevacizumab-ferritin (HFH) fusion protein, in which 9 hRIDs were mutated and could not bind RNA, was reduced compared to the Bevacizumab-ferritin fusion protein (HFH), in which hRID was not mutated hRID(W). In addition, it is the results of confirming that the binding force with hVEGF was improved when the Bevacizumab-ferritin (HFH) fusion protein was formed compared to when only scFv was present. These results show that when RNA is bound to hRID(W), protein folding is improved, and when the Bevacizumab-ferritin fusion protein is formed, the binding force to hVEGF is increased.

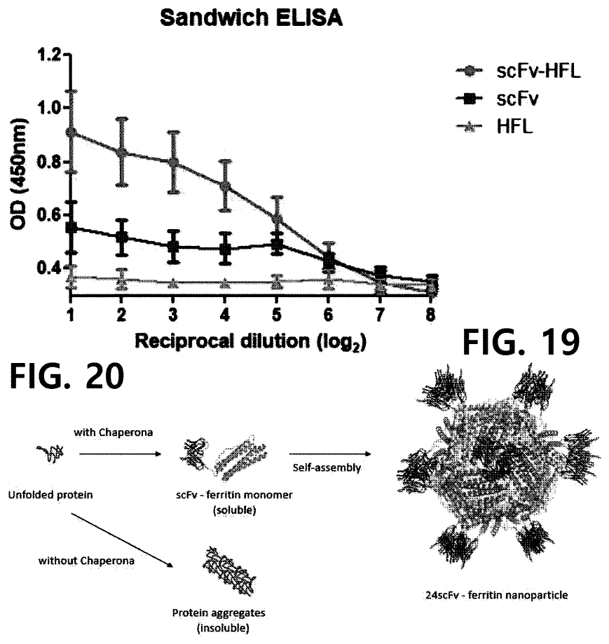

FIG. 18 is a graph comparing the binding affinity of the hRID-Bevacizumab scfv-ferritin (HFL; Human Ferritin light chain) fusion protein by indirect ELISA analysis. FIG. 18 shows the binding affinities of hRID(W)-Bevacizumab scfv-ferritin (HFL) fusion protein nanoparticles, hRID(9m)-Bevacizumab scfv-ferritin (HFL) fusion protein nanoparticles and the folding and targeting of Bevacizumab scfv according to the antibody concentration. FIG. 19 is a graph analyzing the target binding affinity of hRID-scFv-G3SG3TG3SG3-ferritin (scFv-HFL) nanoparticles, hRID-scFv (scFv) and hRID-HFL (HFL) according to an exemplary embodiment of the present invention by sandwich ELISA. The results were obtained by repeating the ELISA three times.

In humans, ferritin exists in two types (HFH/HFL) according to its size. Referring to FIGS. 18 and 19, it can be seen that the fusion protein prepared according to the present invention has an increased affinity for the target by self-assembly regardless of the size/type of ferritin. In addition, according to this example, when it is in the form of hRID-scFv-G3SG3TG3SG3-ferritin (scFv-HFL) fusion protein nanoparticles or Bevacizumab-ferritin (HFH) fusion protein nanoparticles, it can be seen that it bound to more antigens than when 24 times the concentration of hRID-scFv (scFv) or hRID-HFL (HFL) was used. Therefore, it can be seen that the fusion protein of the present invention forms nanoparticles and has a higher binding affinity to the target than when using a monomeric structure (e.g., hRID-scFv (scFv) or hRID-HFL (HFL)) that does not form nanoparticles.

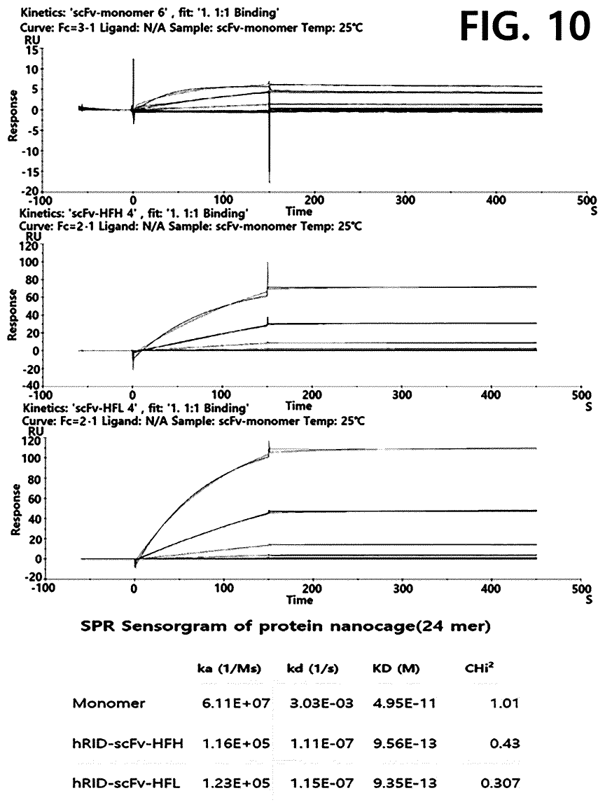

### Example 1-2. ELISAX and SPR Analysis of Trastuzumab-Ferritin Fusion Protein Nanoparticles (FIG. 10)

The binding force between Trastuzumab, which is the prototype of scFv, and HER2 and the binding force between the scFv-ferritin fusion protein nanoparticles and HER2 were compared by using SPR (Surface plasmon resonance) analysis.

Specifically, a Biacore T100 analyzer (GE Healthcare, Uppsala, Sweden) was used, and in the biosensor analysis, HER2 protein (10 μg/mL in 10 mM Sodium acetate, pH 5.0) was immobilized by flowing to a CM5 sensor chip (GE Healthcare, Uppsala, Sweden), which was activated by EDC/NHS (N′-(3-dimethylaminopropyl) carbodiimide hydrochloride/N-hydroxysuccinimide), for 5 minutes at a flow rate of 10 μL/min. The activated portion remaining on the surface of the sensor chip was deactivated by adding 1.0 M ethanolamine (pH 8.5). In this state, after Herceptin or scFv-ferritin fusion protein nanoparticles were diluted at each concentration in a buffer solution (1×PBS, pH 7.4), the binding sensorgram and dissociation sensorgram were determined while flowing at a flow rate of 30 μL/min. In this case, in order to correct sensorgrams caused by non-specific binding to the sensor chip, cells that were completely inactivated only by BSA (bovine serum albumin) were used, and non-specific binding was corrected by subtracting the BSA sensorgram from the HER2 sensorgram. The surface of the sensor chip was regenerated by flowing a regeneration buffer solution (20 mM NaOH).

### Example 2. Migration Test

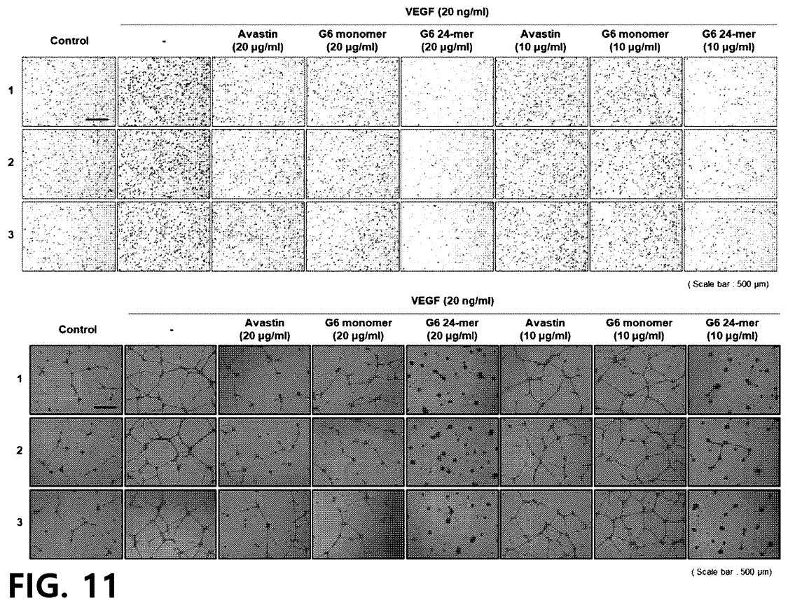

HUVECs were maintained in a serum starvation state for 6 hours, and VEGF, VEGF+Avastin, VEGF+G6 monomer and VEGF+G6 24-mer were treated in the lower portion of transwell plates at the concentrations shown in the drawings, and 1×10⁵ cells were seeded in the transwell. After 5 hours, cells that had migrated to the transwell were identified through H&E staining (top of FIG. 11). Similarly, HUVECs were maintained in a serum starvation state for 6 hours, and 1.3×10⁵ cells were mixed with each of VEGF, VEGF+Avastin, VEGF+G6 monomer and VEGF+G6 24-mer that were treated at the concentrations shown in the drawings on a matrigel-coated plate to confirm angiogenesis 18 hours after seeding (bottom of FIG. 11). The scFv monomer showed the same effect as Avastin at the same concentration on vascular endothelial cell mobility and angiogenesis, and showed a stronger effect than Avastin in the multimer (G6 24-mer). Therefore, when the nanoparticles according to the present invention (in this example, multimer (G6 24-mer)) were treated, it can be confirmed that the inhibitory effect of vascular endothelial cell mobility and angiogenesis to VEGF was superior to that of the VEGF-treated G6 monomer control group.

### Example 3. Confirmation of the Therapeutic Effect of Induced CNV Lesions

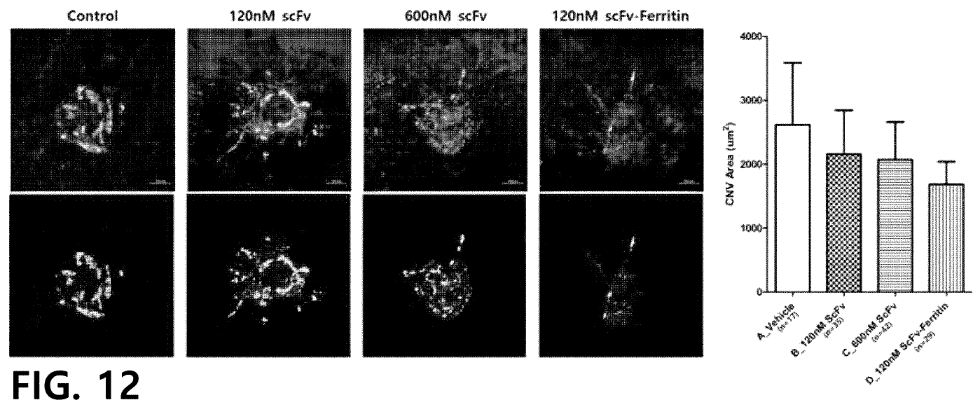

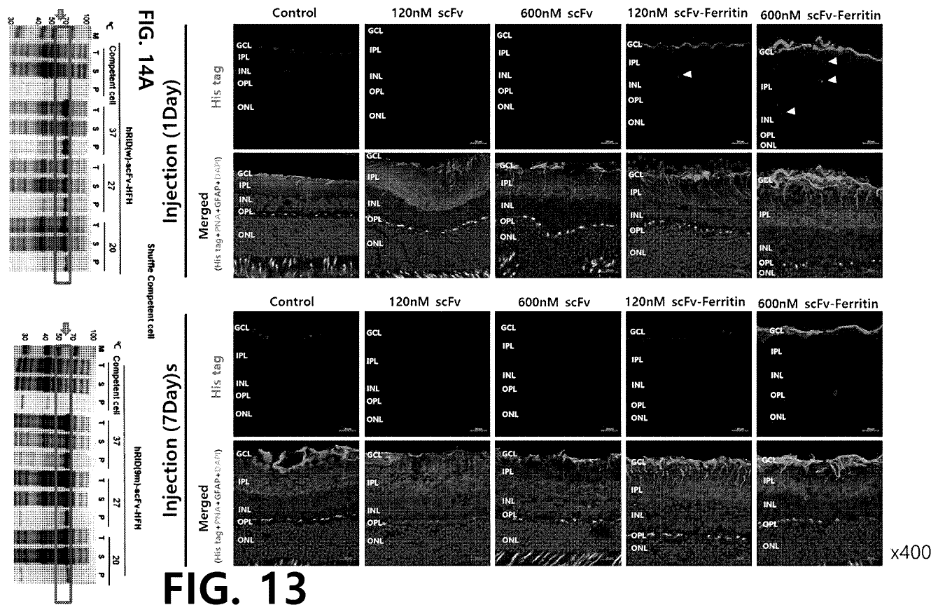

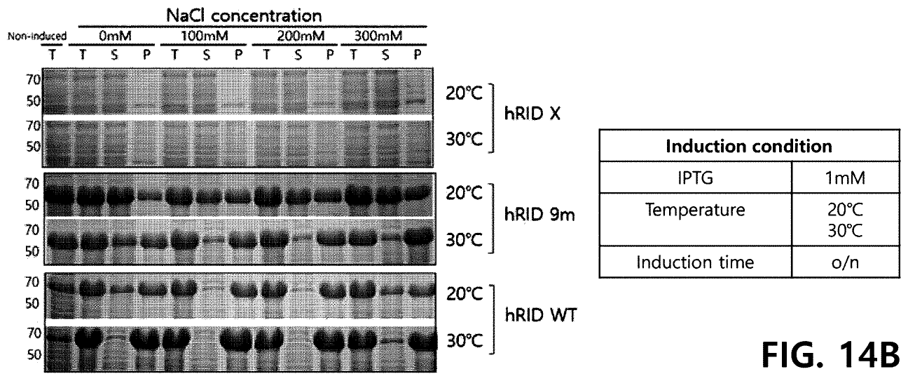

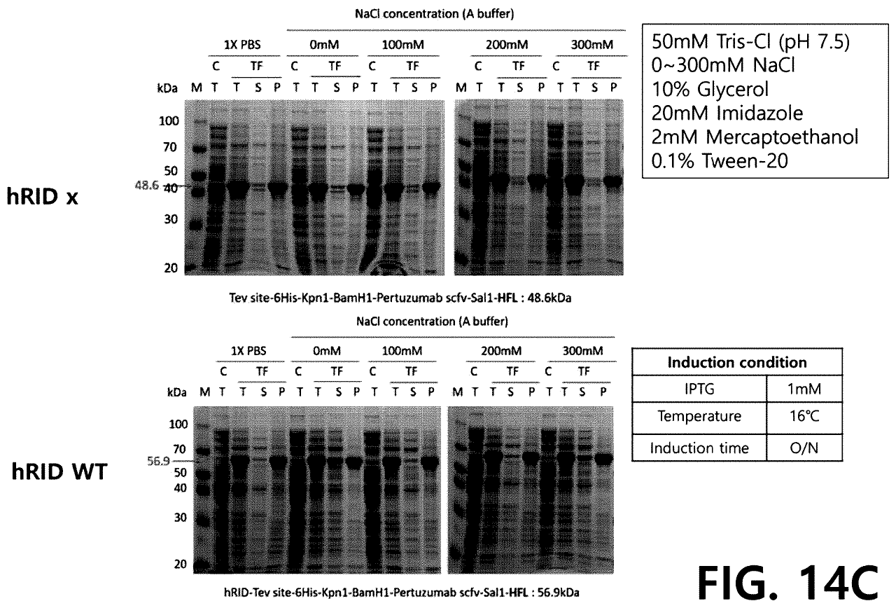

The laser-induced CNV animal model focuses on the fundus retina and retinal pigment layer by using argon green laser, which is ophthalmic laser treatment equipment, on 24 8-week-old males of C57BL/6 mouse to attempt 4 to 5 laser photo coagulations by setting the power of laser at 100 mW. On the day of laser treatment, 4 to 5 animals in each group were intraocularly injected with 4 experimental groups (120 nM ScFv monomer, 600 nM scFv, 120 nM ScFv-Ferritin, 600 nM ScFv-Ferritin) and a normal control group (vehicle), and after 14 days, angiography using fluorescein-dextran was performed, the animals were sacrificed, the eyes were removed, the choroid flatmount was performed, the CNV was observed under a confocal microscope, and the therapeutic effect was analyzed by reducing the CNV size for each group. The average area of choroidal neovascularization decreased in the order of a control group (saline), 120 nM scFv, 600 nM scFv and 120 nM scFv-Ferritin, and the smallest average choroidal neovascularization was observed in the scFv-Ferritin group. This indicates that 120 nM scFv-ferritin nanoparticles have a stronger retinochoroidal vascular inhibition effect than scFv at a concentration of 120 or 600 nM (refer to FIG. 12).

### Example 4. Confocal Microscopy Confirmation of Nanoparticle-Injected Retina

After anesthetizing 10 animal model 8-week-old male C57BL/6 mice, 4 experimental groups (120 nM ScFv monomer, 600 nM scFv, 120 nM ScFv-Ferritin, 600 nM ScFv-Ferritin) and a normal control (vehicle) drug were intravitreously injected into the right eye of anesthetized mice in a volume of 1 μL each by using a Hamilton 32G needle microsyringe. Thereafter, on day 1 and day 7, one animal from each group was sacrificed under anesthesia, the eye was removed, and frozen section was made at 10 μm thickness, and antibodies such as anti-Glial fibrillary acidic protein antibody (Millipore) and Lectin-PNA Alexa 488 (Molecular Probes, Eugene, OR, USA), which is mainly identified in the photoreceptor layer and outer plexiform layer for the purpose of confirming the distribution of astrocytes by counterstaining to confirm the intraretinal structure along with polyclonal His-Tag antibody (Cell Signaling Technology) that can confirm the distribution of ScFv in the retina as follows, were used to perform cross fluorescent staining, and then, the distribution of scFV nanoparticles was determined by intraretinal layer for each group by photographing under a confocal fluorescence microscope (LSM710; Carl Zeiss, Jena, Germany) (refer to FIG. 13). scFv was stained by using His tag antibody (red). In the day 1 image, 120 nM scFv and 600 nM scFv were not observed in the retina, but it was confirmed that 120 nM and 600 nM scFv-Ferritin were distributed in large amounts on the inner layer surface of the retina, and scFv released from the scFv-Ferritin fusion protein nanoparticles penetrated and distributed within the retina (arrowhead). In the day 7 image, whereas 120 nM scFv and 600 nM scFv were not identified in the retina, 120 nM and 600 nM scFv-Ferritin were clearly observed on the surface of the inner retina layer, and scFv within the retina was observed in the 600 nM scFv-Ferritin group. This indicates that compared to the administration of scFv alone, scFv-Ferritin fusion protein nanoparticles adhere to the inner layer surface of the retina at a higher concentration and deliver scFv to the inner surface of the retina and the entire retina for a long period of time, thereby maintaining a strong and continuous therapeutic effect for a longer period of time.

### Example 5. Solubility of Nanoparticles According to hRDI

### Example 5-1. Bevacizumab Fusion Protein Nanoparticles

It is known that RNA functions like molecular chaperones in E. coli, thereby helping to improve protein solubility and folding. Therefore, in order to confirm this, a point mutation (9m) was made to prevent tRNA from binding to the representative hRID. An hRID(w)-Bevacizumab-ferritin firn, which is a wild-type (W) form of hRID, and an hRID(9m)-Bevacizumab-ferritin form, which is a form in which hRID was mutated (9m) to prevent tRNA from binding to hRID, were expressed to compare the water solubility of proteins by running SDS PAGE GEL.

### Example 5-2. Trastuzumab Fusion Protein Nanoparticles

Trastuzumab-ferritin, which is a form in which hRID was not bound, hRID(WT)-Trastuzumab-ferritin, which is a form in which hRID was wild-type (W), and hRID(9m)-Trastuzumab-ferritin, which is a form in which mutations (9m) were made to prevent tRNA binding to hRID were expressed to compare the water solubility of proteins by running SDS PAGE GEL. It was confirmed that whereas the protein to which hRID was not bound was not expressed in E. coli, the protein to which hRID was bound was expressed. In addition, it was confirmed that, even if hRID was bound to the protein, water solubility was very poor if tRNA was not bound to hRID.

### Example 5-3. Pertuzumab Fusion Protein Nanoparticles

Pertuzumab-ferritin, which is a form in which hRID was not bound, and hRID(W)-Pertuzumab-ferritin, which is a form in which hRID was wild-type (W), were expressed to compare the water solubility or proteins by running SDS PAGE gel. Although the protein to which hRID was not bound at all was expressed in E. coli, the water solubility was not increased at any NaCl concentration, but it was confirmed that the protein to which hRID was bound increased the water solubility of the protein according to the NaCl concentration.

### Example 6. Expression and Purification of Proteins According to Binding of hRID(Purification: Ni-Affinity Chromatography and SEC)

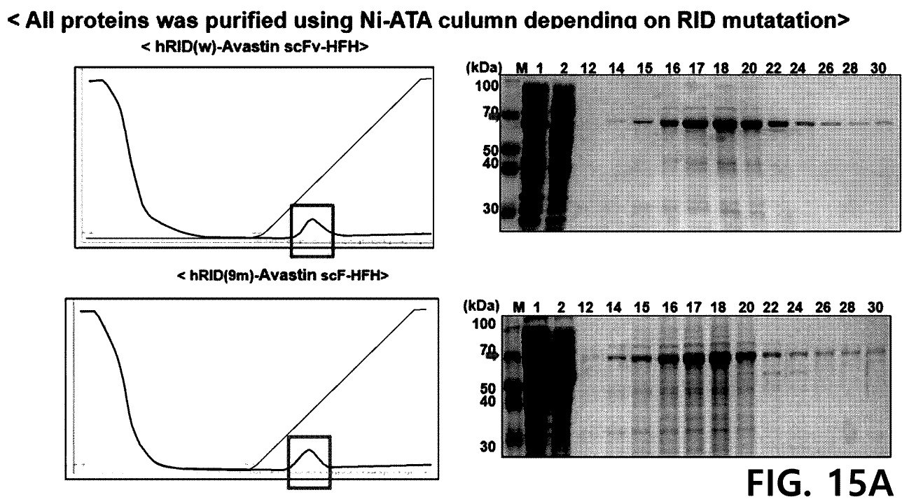

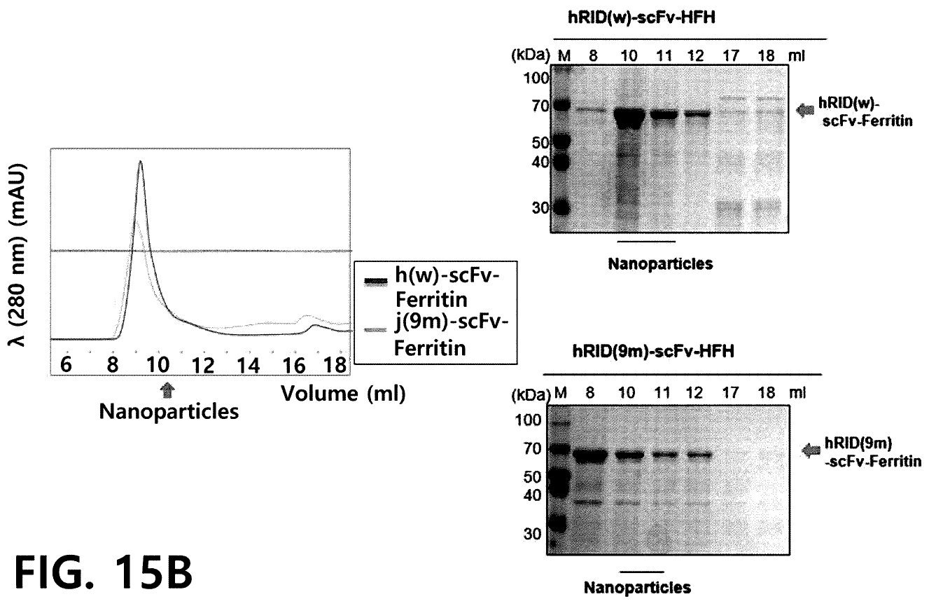

### Example 6-1. Bevacizumab Fusion Protein Nanoparticles (FIGS. 15 a, b)

In order to confirm RNA dependence upon the formation of ferritin nanoparticles, the form of hRID(W)-Bevacizumab-ferritin and the form of hRID(9m)-Bevacizumab-ferritin were expressed, and purification was performed to determine what role RNA binding plays in the formation of nanoparticles.

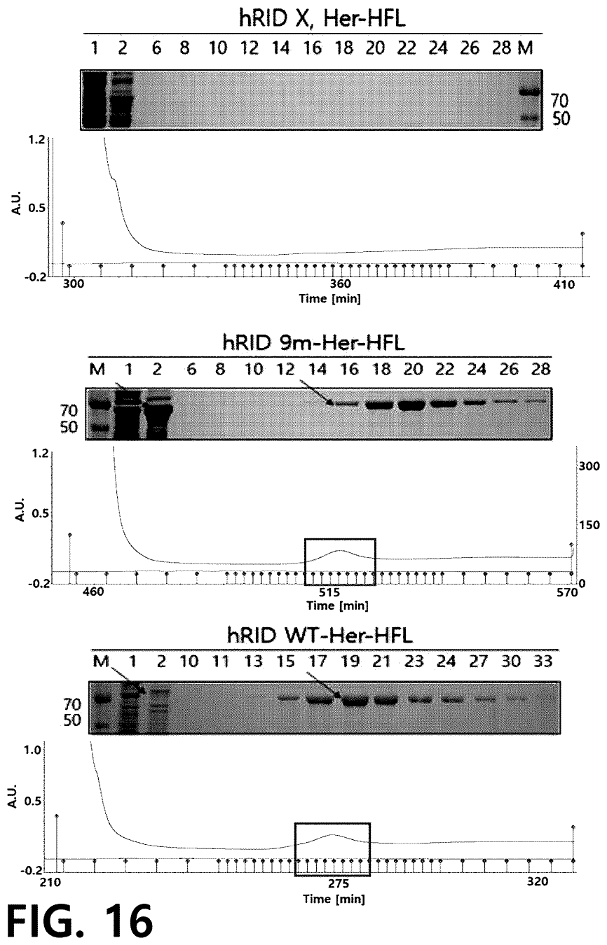

### Example 6-2. Trastuzumab Fusion Protein Nanoparticles (FIG. 16)

In order to confirm RNA dependence upon the formation of ferritin nanoparticles, the forms of Trastuzumab-ferritin and hRID(W)-Trastuzumab-ferritin and the form of hRID(9m)-Trastuzumab-ferritin were expressed to perform purification. It was confirmed that the proteins to which hRID was not bound were not purified.

### Example 7. Assembly of Nanoparticles According to Mutation of hRID

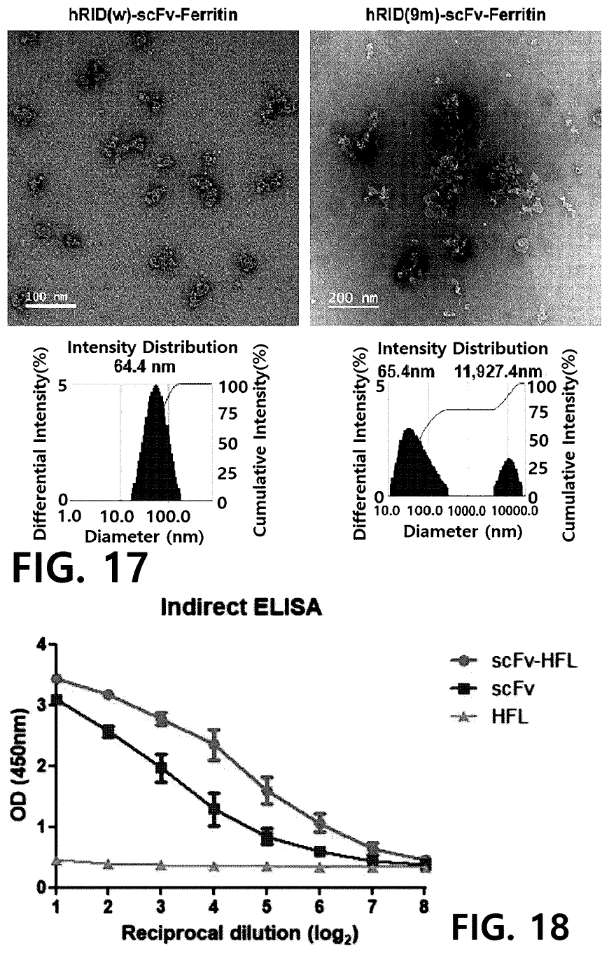

In the case of Bevacizumab, there was no significant difference in the expression and purification process compared to Trastuzumab, in the presence or absence of mutations in hRID, but it was confirmed that when the structure formation was determined by TEM, hRID(W)-Bevacizumab-ferritin formed an appropriate structure, but the structure of hRID(9m)-Bevacizumab-ferritin was irregular and heterogeneous (refer to FIG. 17). In summary, RNA binding prevents aggregation into irregular shapes and plays an important role in nanoparticle assembly into homogenous regular shapes.

[Sequence List Free Text]

SEQ ID NO: 1
hRID(WT) Protein seq
MSEQHAQAAVQAAEVKVDGSEPKLSKNELKRRLKAEKKVAEKEAK
QKELSEKQLSQATAAATNHTTDNGVGPEEESV
SEQ ID NO: 2
hRID(WT) DNA seq
atgtctgaacaacacgcacaggcggccgtgcaggcggccgaggtgaaagtggatggcagcgagccgaa
actgagcaagaatgagctgaagagacgcctgaaagctgagaagaaagtagcagagaaggaggccaaacagaaagag
ctcagtgagaaacagctaagccaagccactgctgctgccaccaaccacaccactgataatggtgtgggtcctgaggaag
agagcgtg
SEQ ID NO: 3
hRID(9m) DNA seq
atgtctgaacaacacgcacaggcggccgtgcaggcggccgaggtgaaagtggatggcagcgagccgaa
actgagcgcgaatgagctggcggcgcgcctggcggctgaggcggcggtagcagaggcggaggccgcgcaggcgg
agctcagtgagaaacagctaagccaagccactgctgctgccaccaaccacaccactgataatggtgtgggtcctgagga
agagagcgtg
SEQ ID NO: 4
Bacterioferritin Protein Seq
Met Lys Gly Asp Thr Lys Val Ile Asn Tyr Leu Asn Lys Leu Leu Gly Asn
Glu Leu Val Ala Ile Asn Gln Tyr Phe Leu His Ala Arg Met Phe Lys Asn Trp Gly
Leu Lys Arg Leu Asn Asp Val Glu Tyr His Glu Ser Ile Asp Glu Met Lys His Ala
Asp Arg Tyr Ile Glu Arg Ile Leu Phe Leu Glu Gly Leu Pro Asn Leu Gln Asp Leu
Gly Lys Leu Asn Ile Gly Glu Asp Val Glu Glu Met Leu Arg Ser Asp Leu Ala Leu
Glu Leu Asp Gly Ala Lys Asn Leu Arg Glu Ala Ile Gly Tyr Ala Asp Ser Val His
Asp Tyr Val Ser Arg Asp Met Met Ile Glu Ile Leu Arg Asp Glu Glu Gly His Ile Asp
Trp Leu Glu Thr Glu Leu Asp Leu Ile Gln Lys Met Gly Leu Gln Asn Tyr Leu Gln
Ala Gln Ile Arg Glu Glu Gly
SEQ ID NO: 5
scfv of Trastuzumab protein seq
M T V A Q A A E V Q L V E S G G G L V Q P G G S L R L S C A A S G
F N I K D T Y I H W V R Q A P G K G L E W V A R I Y P I N G Y T R Y A D S
V K G R F T I S A D T S K N T A Y L Q M N S L R A E D T A V Y Y C S R
W G G D G F Y A M D Y W G Q G T L V T V S G G G G S G G G G S G G G
G S D I Q M T Q S P S S L S A S V G D R V T I T C R A S Q D V N T A V A
W Y Q Q K P G K A P K L L I Y S A S F L Y S G V P S R F S G S R S G T D F T
L T I S S L Q P E D F A T Y Y C Q Q H Y T T P P T F G Q G T K V E I K
scfv of Trastuzumab DNA seq
atgaccgtggcccaggcggccgaagtgcagttagtcgaatccggtggaggcttagttcagcctggtggcag
ccttcgtctgtcatgcgctgcttctggttttaacatcaaagatacttatatccactgggtacggcaggccccaggaaaaggtc
tggaatgggtggcccgtatttatcccactaatggatatacccgttatgccgactcagtaaaaggtcgtttcactatttcggca
gatactagtaaaaatacagcttatctgcaaatgaactctctcagagcagaggataccgcggtttattattgttctcgctggggt
ggtgatggtttctatgctatggattattggggtcaaggtactttggttacggtgtctggtggtggtggttcaggcggaggtgg
tagtggtggtggaggtagcgacattcagatgacgcaatctcctagcagtctttcagcgagtgtcggtgatcgtgtcaccatt
acatgtcgtgcatcacaagatgttaataccgcagtggcgtggtatcagcagaagccgggtaaagcaccgaaattgctgat
atattccgcctcattcttatatagcggtgttccttctcgctttagcggatcgcgaagcggaacagactttaccctgacaatatc
gtctctgcagccggaggattttgcaacgtattattgccaacagcattatacaaccccaccgacctttggtcagggtacgaaa
gtagaaattaag
SEQ ID NO: 6
Scfv of Bevacizumab protein seq
MEvqlvesggglvqpggslrlscaasgftisdywihwvrqapgkglewvagitpaggytyyadsvkgr
ftisadtskntaylqmnslraedtavyycarfvfflpyamdywgqgtlvtvssggggsggggsggggsdiqmtqsps
slsasvgdrvtitcrasqdvstavawyqqkpgkapklliysasflysgvpsrfsgsgsgtdftltisslqpedfatyycqq
syttpptfgqgtkveikrtv
Scfv of Bevacizumab DNA seq
ATG GAA GTC CAG TTG GTG GAG TCC GGT GGT GGC CTG GTC
CAA CCG GGT GGG AGT CTC AGG CTC TCC TGC GCC GCT TCA GGG TTC
ACA ATC TCT GAC TAT TGG ATT CAC TGG GTG CGC CAG GCG CCA GGC
AAG GGG CTG GAG TGG GTC GCG GGC ATT ACC CCG GCG GGC GGC
TAC ACC TAT TAC GCC GAC TCA GTC AAG GGC CGC TTC ACA ATC TCC
GCG GAC ACC AGC AAG AAC ACC GCG TAC CTC CAA ATG AAC TCG
CTG CGC GCC GAA GAC ACC GCC GTG TAC TAT TGC GCG AGA TTC GTG
TTC TTC CTG CCG TAC GCC ATG GAC TAC TGG GGC CAG GGA ACG CTG
GTG ACC GTG AGT AGT GGT GGT GGT GGT AGC GGT GGA GGT GGT
AGT GGT GGT GGT GGT TCC GAT ATC CAG ATG ACA CAG TCC CCC AGT
TCA CTG TCC GCG TCC GTT GGG GAC CGT GTC ACC ATT ACC TGT CGC
GCC AGT CAA GAC GTT AGC ACG GCG GTC GCG TGG TAC CAG CAA
AAG CCA GGG AAG GCG CCA AAA CTG CTG ATC TAT AGC GCG AGC
TTC CTC TAC AGC GGA GTC CCC AGT AGG TTC TCC GGA TCG GGG TCC
GGC ACG GAC TTC ACC TTG ACC ATC TCC AGC CTC CAG CCG GAG GAT
TTC GCT ACG TAC TAC TGC CAG CAG AGC TAC ACG ACG CCG CCC ACC
TTC GGC CAA GGC ACC AAG GTG GAG ATA AAA CGT ACG GTT
SEQ ID NO: 7
Scfv of pertuzumab protein seq
EVQLVESGGGLVQPGGSLRLSCAASGFTFTDYTMDWVRQAPGKGLE
WVADVNPNSGGSIYNQRFKGRFTLSVDRSKNTLYLQMNSLRAEDTAVYYC
ARNLGPSFYFDYWGQGTLVTVSSGGGGSGGGGSGGGGSDIQMTQSPSSLSA
SVGDRVTITCKASQDVSIGVAWYQQKPGKAPKLLIYSASYRYTGVPSRFSGS
GSGTDFTLTISSLQPEDFATYYCQQYYIYPYTFGQGTKVEIKRT
Scfv of pertuzumab DNA seq
GAAGTTCAGCTGGTTGAAAGCGGCGGTGGTCTGGTTCAGCCGGGT
GGTTCTCTGCGTCTGTCTTGTGCTGCTAGCGGTTTCACCTTTACTGATTAC
ACCATGGATTGGGTTCGTCAGGCGCCGGGTAAAGGTCTGGAATGGGTTG
CTGATGTGAACCCGAACTCTGGTGGTAGCATCTACAACCAGCGTTTCAAA
GGTCGTTTTACCCTGTCTGTTGATCGTTCTAAAAACACCCTGTACCTGCAG
ATGAACAGCCTGCGTGCGGAAGATACCGCTGTATATTACTGTGCACGTAA
CCTGGGCCCGTCTTTTTATTTTGATTATTGGGGTCAGGGTACTCTGGTTAC
CGTTAGCTCTGGTGGTGGCGGTAGCGGCGGCGGTGGTAGCGGTGGTGGT
GGTTCTGATATTCAAATGACCCAGTCTCCAAGCAGCCTGTCTGCTAGCGT
TGGTGATCGTGTTACCATCACTTGTAAAGCATCCCAGGATGTTTCTATCG
GTGTTGCGTGGTATCAACAGAAACCGGGTAAAGCACCGAAATTACTGAT
CTATTCCGCGTCTTATCGTTATACTGGCGTTCCGTCTCGTTTCTCTGGTTCT
GGTAGCGGCACTGATTTCACCCTGACCATTTCTAGCCTGCAGCCGGAAGA
TTTCGCTACCTATTATTGCCAGCAGTATTATATTTACCCGTATACCTTCGG
TCAGGGCACCAAAGTTGAAATTAAACGTACC
SEQ ID NO: 8
Linker 1 Protein seq
SGGGGSGGGG
SEQ ID NO: 9
Linker 2 Protein seq
GGGGSGGGGSGGGGSGQAGQHAAGSGSGSS
SEQ ID NO: 10
Linker 3 Protein seq
GGGSGGGTGGGSGGG
SEQ ID NO: 11
hRID-scfv of Trastuzumab-FR Vector DNA seq
gtggaggtggttccggtggtggtggtagtggtggaggtggtagtggccaggccggccagcacgcggccg
gcagcggatccggcagcagcggcagcagcagctcccagattcgtcagaattattccaccgacgtggaggcagccgtca
acagcctggtcaatttgtacctgcaggcctcctacacctacctctctctgggcttctatttcgaccgcgatgatgtggctctgg
aaggcgtgagccacttcttccgcgaactggccgaggagaagcgcgagggctacgagcgtctcctgaagatgcaaaacc
agcgtggcggccgcgctctcttccaggacatcaagaagccagctgaagatgagtggggtaaaaccccagacgccatga
aagctgccatggccctggagaaaaagctgaaccaggcccttttggatcttcatgccctgggttctgcccgcacggacccc
catctctgtgacttcctggagactcacttcctagatgaggaagtgaagcttatcaagaagatgggtgaccacctgaccaac
ctccacaggctgggtggcccggaggctgggctgggcgagtatctcttcgaaaggctcactctcaagcacgactaa
SEQ ID NO: 12
hRID-scfv of Bevacizumab-FR Vector DNA seq
gtggttcaggtggtggtggtacgaccgcgtccacctcgcaggtgcgccagaactaccaccaggactcagag
gccgccatcaaccgccagatcaacctggagctctacgcctcctacgtttacctgtccatgtcttactactttgaccgcgatga
tgtggctttgaagaactttgccaaatactttcttcaccaatctcatgaggagagggaacatgctgagaaactgatgaagctg
cagaaccaacgaggtggccgaatcttccttcaggatatcaagaaaccagactgtgatgactgggagagcgggctgaatg
caatggagtgtgcattacatttggaaaaaaatgtgaatcagtcactactggaactgcacaaactggccactgacaaaaatg
acccccatttgtgtgacttcattgagacacattacctgaatgagcaggtgaaagccatcaaagaattgggtgaccacgtga
ccaacttgcgcaagatgggagcgcccgaatctggcttggcggaatatctctttgacaagcacaccctgggagacagtgat
aatgaaagctga
SEQ ID NO: 13
hRID-scvf of pertuzumab-FR Vector DNA seq
gcggtggtggtaccggtggtggtagcggtggtggtgtcgacatgagctctcagattcgtcagaactactcca
ccgacgtggaggcagcagttaacagcctggtcaacctgtacctgcaagcatcctacacctacctgtccctgggtttctactt
cgaccgtgacgacgtagctctggaaggtgtttctcacttcttccgcgagctggcagaagagaaacgtgaaggttatgaac
gcctgctgaaaatgcagaaccagcgcggtggtcgtgctctgttccaggacatcaagaaaccagctgaagatgaatgggg
caaaaccccggatgccatgaaagctgccatggcgctggagaagaaactgaaccaggcgctgctggatctgcatgcgct
gggttctgcgcgtactgatcctcatctgtgcgatttcctggaaactcactttctggatgaagaagtgaaactgatcaagaaaa
tgggcgaccacctgaccaatctgcaccgtctgggcggcccggaaggggcctgggcgaatatctgtttgaacgtctgac
gctgaaacacgactaaaagctt

1. A fusion protein, comprising:
a ferritin protein;
a single-chain variable fragment which is bound to an N-terminus of the ferritin protein; and
an N-terminus domain (hLysRS N-terminal appended RNA interacting domain; hRID) which is isolated from a human-derived lysyl tRNA synthetase that is bound to an N-terminus of the single-chain variable fragment.

2. The fusion protein of claim 1, wherein the single-chain variable fragment is derived from any one antibody selected from the group consisting of Trastuzumab, Bevacizumab and Pertuzumab.

3. The fusion protein of claim 1, wherein the hRID is represented by the amino acid sequence of SEQ ID NO: 1.

4. The fusion protein of claim 1, wherein the ferritin protein is represented by the amino acid sequence of SEQ ID NO: 4.

5. The fusion protein of claim 1, wherein the fusion protein further comprises a linker protein between the single-chain variable fragment (scFv) and the ferritin protein.

6. The fusion protein of claim 5, wherein the linker protein has an amino acid sequence represented by any one of the amino acid sequences of SEQ ID NOs: 8 to 10.

7. Nanoparticles, which are formed by self-assembly of 2 to 24 pieces of the fusion protein of claim 1 as a monomer.

8. A polynucleotide, which encodes the fusion protein of claim 1.

9. A recombination expression vector, comprising the polynucleotide of claim 8.

10. A host cell, which is transformed by the expression vector according to claim 9.

11. The host cell of claim 10, wherein the host cell is selected from the group consisting of Escherichia bacteria, Bacillus bacteria, Pseudomonas bacteria, lactic acid bacteria, yeast, animal cells and insect cells.

12. A method for preparing nanoparticles, comprising the steps of:
(a) preparing an expression vector comprising a polynucleotide encoding a fusion protein, which consists of a ferritin protein; a single-chain variable fragment which is bound to an N-terminus of the ferritin protein; and an N-terminus domain (hLysRS N-terminal appended RNA interacting domain; hRID) which is isolated from a human-derived lysyl tRNA synthetase that is bound to an N-terminus of the single-chain variable fragment;
(b) preparing a transformant by introducing the expression vector into a host cell;
(c) culturing the transformant to induce expression of the fusion protein; and
(d) purifying nanoparticles formed by self-assembly of the expressed fusion protein.

13. The method of claim 12, wherein the nanoparticles are formed by self-assembly of 2 to 24 pieces of the fusion protein monomer.

14. The method of claim 12, wherein the single-chain variable fragment is derived from any one antibody selected from the group consisting of Trastuzumab, Bevacizumab and Pertuzumab.

15. A pharmaceutical composition for ameliorating or treating a disease, comprising the fusion protein of claim 1.

16. Use of nanoparticles in the treatment of a target disease, wherein the nanoparticles are formed by self-assembly of 2 to 24 pieces of a fusion protein, which comprises a ferritin protein; a single-chain variable fragment which is bound to an N-terminus of the ferritin protein; and an N-terminus domain (hLysRS N-terminal appended RNA interacting domain; hRID) which is isolated from a human-derived lysyl tRNA synthetase that is bound to an N-terminus of the single-chain variable fragment, as a monomer.


SEQUENCE LISTING (13 sequences)

SEQ ID NO: 1 - protein, 77 aa, Artificial Sequence
  Description of Artificial Sequence Synthetic polypeptide
  hRID(WT) Protein seq
MSEQHAQAAVQAAEVKVDGSEPKLSKNELKRRLKAEKKVAEKEAKQKELSEKQLSQATAAATNHTTDNGVGPEEESV

SEQ ID NO: 2 - DNA, 231 bp, Artificial Sequence
  Description of Artificial Sequence Synthetic polynucleotide
  hRID(WT) DNA seq
atgtctgaacaacacgcacaggcggccgtgcaggcggccgaggtgaaagtggatggcagcgagccgaaactgagcaagaatgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtg

SEQ ID NO: 3 - DNA, 231 bp, Artificial Sequence
  Description of Artificial Sequence Synthetic polynucleotide
  hRID(9m) DNA seq
atgtctgaacaacacgcacaggcggccgtgcaggcggccgaggtgaaagtggatggcagcgagccgaaactgagcgcgaatgagctggcggcgcgcctggcggctgaggcggcggtagcagaggcggaggccgcgcaggcggagctcagtgagaaacagctaagccaagccactgctgctgccaccaaccacaccactgataatggtgtgggtcctgaggaagagagcgtg

SEQ ID NO: 4 - protein, 158 aa, Artificial Sequence
  Description of Artificial Sequence Synthetic polypeptide
  Bacterioferritin Protein Seq
MKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEG

SEQ ID NO: 5 - DNA, 744 bp, Artificial Sequence
  Description of Artificial Sequence Synthetic polynucleotide
  scfv of Trastuzumab DNA seq
atgaccgtggcccaggcggccgaagtgcagttagtcgaatccggtggaggcttagttcagcctggtggcagccttcgtctgtcatgcgctgcttctggttttaacatcaaagatacttatatccactgggtacggcaggccccaggaaaaggtctggaatgggtggcccgtatttatcccactaatggatatacccgttatgccgactcagtaaaaggtcgtttcactatttcggcagatactagtaaaaatacagcttatctgcaaatgaactctctcagagcagaggataccgcggtttattattgttctcgctggggtggtgatggtttctatgctatggattattggggtcaaggtactttggttacggtgtctggtggtggtggttcaggcggaggtggtagtggtggtggaggtagcgacattcagatgacgcaatctcctagcagtctttcagcgagtgtcggtgatcgtgtcaccattacatgtcgtgcatcacaagatgttaataccgcagtggcgtggtatcagcagaagccgggtaaagcaccgaaattgctgatatattccgcctcattcttatatagcggtgttccttctcgctttagcggatcgcgaagcggaacagactttaccctgacaatatcgtctctgcagccggaggattttgcaacgtattattgccaacagcattatacaaccccaccgacctttggtcagggtacgaaagtagaaattaag

SEQ ID NO: 6 - DNA, 738 bp, Artificial Sequence
  Description of Artificial Sequence Synthetic polynucleotide
  Scfv of Bevacizumab DNA seq
atggaagtccagttggtggagtccggtggtggcctggtccaaccgggtgggagtctcaggctctcctgcgccgcttcagggttcacaatctctgactattggattcactgggtgcgccaggcgccaggcaaggggctggagtgggtcgcgggcattaccccggcgggcggctacacctattacgccgactcagtcaagggccgcttcacaatctccgcggacaccagcaagaacaccgcgtacctccaaatgaactcgctgcgcgccgaagacaccgccgtgtactattgcgcgagattcgtgttcttcctgccgtacgccatggactactggggccagggaacgctggtgaccgtgagtagtggtggtggtggtagcggtggaggtggtagtggtggtggtggttccgatatccagatgacacagtcccccagttcactgtccgcgtccgttggggaccgtgtcaccattacctgtcgcgccagtcaagacgttagcacggcggtcgcgtggtaccagcaaaagccagggaaggcgccaaaactgctgatctatagcgcgagcttcctctacagcggagtccccagtaggttctccggatcggggtccggcacggacttcaccttgaccatctccagcctccagccggaggatttcgctacgtactactgccagcagagctacacgacgccgcccaccttcggccaaggcaccaaggtggagataaaacgtacggtt

SEQ ID NO: 7 - DNA, 729 bp, Artificial Sequence
  Description of Artificial Sequence Synthetic polynucleotide
  Scfv of pertuzumab DNA seq
gaagttcagctggttgaaagcggcggtggtctggttcagccgggtggttctctgcgtctgtcttgtgctgctagcggtttcacctttactgattacaccatggattgggttcgtcaggcgccgggtaaaggtctggaatgggttgctgatgtgaacccgaactctggtggtagcatctacaaccagcgtttcaaaggtcgttttaccctgtctgttgatcgttctaaaaacaccctgtacctgcagatgaacagcctgcgtgcggaagataccgctgtatattactgtgcacgtaacctgggcccgtctttttattttgattattggggtcagggtactctggttaccgttagctctggtggtggcggtagcggcggcggtggtagcggtggtggtggttctgatattcaaatgacccagtctccaagcagcctgtctgctagcgttggtgatcgtgttaccatcacttgtaaagcatcccaggatgtttctatcggtgttgcgtggtatcaacagaaaccgggtaaagcaccgaaattactgatctattccgcgtcttatcgttatactggcgttccgtctcgtttctctggttctggtagcggcactgatttcaccctgaccatttctagcctgcagccggaagatttcgctacctattattgccagcagtattatatttacccgtataccttcggtcagggcaccaaagttgaaattaaacgtacc

SEQ ID NO: 8 - protein, 10 aa, Artificial Sequence
  Description of Artificial Sequence Synthetic peptide
  Linker 1 Protein seq
SGGGGSGGGG

SEQ ID NO: 9 - protein, 30 aa, Artificial Sequence
  Description of Artificial Sequence Synthetic polypeptide
  Linker 2 Protein seq
GGGGSGGGGSGGGGSGQAGQHAAGSGSGSS

SEQ ID NO: 10 - protein, 15 aa, Artificial Sequence
  Description of Artificial Sequence Synthetic peptide
  Linker 3 Protein seq
GGGSGGGTGGGSGGG

SEQ ID NO: 11 - DNA, 1644 bp, Artificial Sequence
  Description of Artificial Sequence Synthetic polynucleotide
  hRID- scfv of Trastuzumab-FR Vector DNA seq
atgtctgaacaacacgcacaggcggccgtgcaggcggccgaggtgaaagtggatggcagcgagccgaaactgagcaagaatgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtggaaaacctgtattttcagggtcatcaccatcaccatcacggtaccatgaccgtggcccaggcggccgaagtgcagttagtcgaatccggtggaggcttagttcagcctggtggcagccttcgtctgtcatgcgctgcttctggttttaacatcaaagatacttatatccactgggtacggcaggccccaggaaaaggtctggaatgggtggcccgtatttatcccactaatggatatacccgttatgccgactcagtaaaaggtcgtttcactatttcggcagatactagtaaaaatacagcttatctgcaaatgaactctctcagagcagaggataccgcggtttattattgttctcgctggggtggtgatggtttctatgctatggattattggggtcaaggtactttggttacggtgtctggtggtggtggttcaggcggaggtggtagtggtggtggaggtagcgacattcagatgacgcaatctcctagcagtctttcagcgagtgtcggtgatcgtgtcaccattacatgtcgtgcatcacaagatgttaataccgcagtggcgtggtatcagcagaagccgggtaaagcaccgaaattgctgatatattccgcctcattcttatatagcggtgttccttctcgctttagcggatcgcgaagcggaacagactttaccctgacaatatcgtctctgcagccggaggattttgcaacgtattattgccaacagcattatacaaccccaccgacctttggtcagggtacgaaagtagaaattaagggtggaggtggttccggtggtggtggtagtggtggaggtggtagtggccaggccggccagcacgcggccggcagcggatccggcagcagcggcagcagcagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaactggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaa

SEQ ID NO: 12 - DNA, 1590 bp, Artificial Sequence
  Description of Artificial Sequence Synthetic polynucleotide
  hRID- scfv of Bevacizumab-FR Vector DNA seq
atgtctgaacaacacgcacaggcggccgtgcaggcggccgaggtgaaagtggatggcagcgagccgaaactgagcaagaatgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtggaaaacctgtattttcagggccatcatcatcatcatcatggatccgaagtccagttggtggagtccggtggtggcctggtccaaccgggtgggagtctcaggctctcctgcgccgcttcagggttcacaatctctgactattggattcactgggtgcgccaggcgccaggcaaggggctggagtgggtcgcgggcattaccccggcgggcggctacacctattacgccgactcagtcaagggccgcttcacaatctccgcggacaccagcaagaacaccgcgtacctccaaatgaactcgctgcgcgccgaagacaccgccgtgtactattgcgcgagattcgtgttcttcctgccgtacgccatggactactggggccagggaacgctggtgaccgtgagtagtggtggtggtggtagcggtggaggtggtagtggtggtggtggttccgatatccagatgacacagtcccccagttcactgtccgcgtccgttggggaccgtgtcaccattacctgtcgcgccagtcaagacgttagcacggcggtcgcgtggtaccagcaaaagccagggaaggcgccaaaactgctgatctatagcgcgagcttcctctacagcggagtccccagtaggttctccggatcggggtccggcacggacttcaccttgaccatctccagcctccagccggaggatttcgctacgtactactgccagcagagctacacgacgccgcccaccttcggccaaggcaccaaggtggagataaaacgtacggtttcgggtggaggtggttcaggtggtggtggtacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctga

SEQ ID NO: 13 - DNA, 1596 bp, Artificial Sequence
  Description of Artificial Sequence Synthetic polynucleotide
  hRID- scvf of pertuzumab-FR Vector DNA seq
atgtctgaacaacacgcacaggcggccgtgcaggcggccgaggtgaaagtggatggcagcgagccgaaactgagcaagaatgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtggaaaacctgtattttcagggccatcatcatcatcatcatggtaccggatccgaagttcagctggttgaaagcggcggtggtctggttcagccgggtggttctctgcgtctgtcttgtgctgctagcggtttcacctttactgattacaccatggattgggttcgtcaggcgccgggtaaaggtctggaatgggttgctgatgtgaacccgaactctggtggtagcatctacaaccagcgtttcaaaggtcgttttaccctgtctgttgatcgttctaaaaacaccctgtacctgcagatgaacagcctgcgtgcggaagataccgctgtatattactgtgcacgtaacctgggcccgtctttttattttgattattggggtcagggtactctggttaccgttagctctggtggtggcggtagcggcggcggtggtagcggtggtggtggttctgatattcaaatgacccagtctccaagcagcctgtctgctagcgttggtgatcgtgttaccatcacttgtaaagcatcccaggatgtttctatcggtgttgcgtggtatcaacagaaaccgggtaaagcaccgaaattactgatctattccgcgtcttatcgttatactggcgttccgtctcgtttctctggttctggtagcggcactgatttcaccctgaccatttctagcctgcagccggaagatttcgctacctattattgccagcagtattatatttacccgtataccttcggtcagggcaccaaagttgaaattaaacgtaccggtggtggtagcggtggtggtaccggtggtggtagcggtggtggtgtcgacatgagctctcagattcgtcagaactactccaccgacgtggaggcagcagttaacagcctggtcaacctgtacctgcaagcatcctacacctacctgtccctgggtttctacttcgaccgtgacgacgtagctctggaaggtgtttctcacttcttccgcgagctggcagaagagaaacgtgaaggttatgaacgcctgctgaaaatgcagaaccagcgcggtggtcgtgctctgttccaggacatcaagaaaccagctgaagatgaatggggcaaaaccccggatgccatgaaagctgccatggcgctggagaagaaactgaaccaggcgctgctggatctgcatgcgctgggttctgcgcgtactgatcctcatctgtgcgatttcctggaaactcactttctggatgaagaagtgaaactgatcaagaaaatgggcgaccacctgaccaatctgcaccgtctgggcggcccggaagcgggcctgggcgaatatctgtttgaacgtctgacgctgaaacacgactaaaagctt